(19) United States
(12) Patent Application Publication
(10) Pub. No.: US 2025/0136712 A1
(43) Pub. Date: May 1, 2025

(54) TREATMENT AND PREVENTION OF CANCER USING HER3 ANTIGEN-BINDING MOLECULES

(71) Applicant: Hummingbird Bioscience Pte. Ltd., Singapore (SG)

(72) Inventors: Jerome Douglas Boyd-Kirkup, Singapore (SG); Shreya kar, Singapore (SG); Konrad Paszkiewicz, Singapore (SG); Dipti Thakkar, Singapore (SG); Piers Ingram, Singapore (SG)

(73) Assignee: Hummingbird Bioscience Pte. Ltd., Singapore (SG)

(21) Appl. No.: 18/683,155

(22) PCT Filed: Aug. 12, 2022
(86) PCT No.: PCT/EP2022/072674

§ 371 Date: Feb. 12, 2024

Related U.S. Application Data
(60) Provisional application No. 63/232,883, filed on Aug. 13, 2021.

(51) Int. Cl. C07K 16/32; A61K 39/00; A61P 35/00; C12Q 1/6886

(52) U.S. Cl. CPC C07K 16/32; A61P 35/00; C12Q 1/6886; A61K 2039/505; C07K 2317/565; C07K 2317/73; C12Q 2600/106; C12Q 2600/156

(57) ABSTRACT

The present disclosure provides the use of an antigen-binding molecule that binds to HERS for the treatment or prevention of cancers comprising (i) at least one gene encoding a positive regulator of HERS-mediated signalling that does not comprise an activating mutation; or (ii) at least one gene encoding a negative regulator of HERS-mediated signalling that does not comprise an inactivating mutation.

This application claims priority from U.S. 63/232,883 filed 13 Aug. 2021, the contents and elements of which are herein incorporated by reference for all purposes.

### TECHNICAL FIELD

The present disclosure relates to the fields of molecular biology, more specifically antibody technology and methods of medical treatment and prophylaxis.

### BACKGROUND

HER3 activation has emerged as an important mechanism for both tumor progression and acquired resistance to standard of care therapies in multiple indications. HER3 targeting approaches to date have not shown the expected clinical efficacy. Suboptimal inhibition of HER3-mediated signalling is one possible explanation.

### SUMMARY

In a first aspect, the present disclosure provides an antigen-binding molecule which binds to HER3 for use in a method of treating or preventing a HER3-associated cancer in a subject, wherein the HER3-associated cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

Also provided is the use of an antigen-binding molecule which binds to HER3 in the manufacture of a medicament for use in treating or preventing a HER3-associated cancer in a subject, wherein the HER3-associated cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

Also provided is a method of treating or preventing a HER3-associated cancer in a subject, comprising administering to the subject a therapeutically- or prophylactically-effective amount of an antigen-binding molecule which binds to HER3, wherein the HER3-associated cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

In some embodiments in accordance with the various aspects of the present disclosure, the HER3-associated cancer: (i) does not comprise an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5 and BRAF; or (ii) does not comprise an inactivating mutation to a gene selected from: PTEN, PPP2CA, PIK3R1, PIK3R2, NF1, BAD and PHLPP1. In some embodiments in accordance with the various aspects of the present disclosure, the HER3-associated cancer: (i) does not comprise an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5; or (ii) does not comprise an inactivating mutation to a gene selected from: PTEN, PPP2CA, PIK3R1, PIK3R2, NF1, BAD and PHLPP1.

In some embodiments, the HER3-associated cancer: (i) does not comprise an activating mutation to KRAS; or (ii) does not comprise an activating mutation to PIK3CA; or (iii) does not comprise an activating mutation to BRAF; or (iv) does not comprise an inactivating mutation to PTEN. In some embodiments, the HER3-associated cancer: (i) does not comprise an activating mutation to KRAS; or (ii) does not comprise an activating mutation to PIK3CA; or (iii) does not comprise an inactivating mutation to PTEN.

In some embodiments, the HER3-associated cancer: (i) does not comprise an activating mutation to KRAS and does not comprise an activating mutation to PIK3CA; or (ii) does not comprise an activating mutation to KRAS and does not comprise an inactivating mutation to PTEN; or (iii) does not comprise an activating mutation to PIK3CA and does not comprise an inactivating mutation to PTEN; or (iv) does not comprise an activating mutation to KRAS and does not comprise an activating mutation to BRAF; or (v) does not comprise an activating mutation to PIK3CA and does not comprise an activating mutation to BRAF; or (vi) does not comprise an activating mutation to BRAF and does not comprise an inactivating mutation to PTEN; or (vii) does not comprise an activating mutation to KRAS and does not comprise an activating mutation to PIK3CA, and does not comprise an activating mutation to BRAF; or (viii) does not comprise an activating mutation to PIK3CA and does not comprise an activating mutation to BRAF, and does not comprise an inactivating mutation to PTEN; or (ix) does not comprise an activating mutation to BRAF and does not comprise an activating mutation to KRAS, and does not comprise an inactivating mutation to PTEN; or (x) does not comprise an activating mutation to KRAS and does not comprise an activating mutation to PIK3CA, and does not comprise an inactivating mutation to PTEN; or (xi) does not comprise an activating mutation to KRAS and does not comprise an activating mutation to BRAF and does not comprise an activating mutation to PIK3CA, and does not comprise an inactivating mutation to PTEN. In some embodiments, the HER3-associated cancer: (i) does not comprise an activating mutation to KRAS and does not comprise an activating mutation to PIK3CA; or (ii) does not comprise an activating mutation to KRAS and does not comprise an inactivating mutation to PTEN; or (iii) does not comprise an activating mutation to PIK3CA and does not comprise an inactivating mutation to PTEN; or (iv) does not comprise an activating mutation to KRAS and does not comprise an activating mutation to PIK3CA, and does not comprise an inactivating mutation to PTEN.

In some embodiments, the HER3-associated cancer does not comprise a mutation resulting in upregulation of HER3-mediated signalling.

In some embodiments, the HER3-associated cancer comprises cells expressing NRG1 at a level which is greater than the level of expression by equivalent non-cancerous cells.

Also provided is an antigen-binding molecule which binds to HER3 for use in a method of treating or preventing a HER3-associated cancer in a subject, wherein the HER3-associated cancer comprises a mutation resulting in upregulation of HER3-mediated signalling, and wherein the method further comprises administering an antagonist of HER3-mediated signalling.

Also provided is the use of an antigen-binding molecule which binds to HER3 in the manufacture of a medicament for use in treating or preventing a HER3-associated cancer in a subject, wherein the HER3-associated cancer comprises a mutation resulting in upregulation of HER3-mediated signalling, and wherein the method further comprises administering an antagonist of HER3-mediated signalling.

Also provided is a method of treating or preventing a HER3-associated cancer in a subject, comprising administering to the subject a therapeutically- or prophylactically-effective amount of an antigen-binding molecule which binds to HER3, wherein the HER3-associated cancer comprises a mutation resulting in upregulation of HER3-mediated signalling, and wherein the method further comprises administering an antagonist of HER3-mediated signalling.

In some embodiments, the mutation resulting in upregulation of HER3-mediated signalling is an activating mutation to a gene encoding a positive regulator of HER3-mediated signalling or an inactivating mutation to a gene encoding a negative regulator of HER3-mediated signalling.

In some embodiments in accordance with the various aspects of the present disclosure, the HER3-associated cancer comprises: (i) an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5 and BRAF; and/or (ii) an inactivating mutation to a gene selected from: PTEN, PPP2CA, PIK3R1, PIK3R2, NF1, BAD and PHLPP1. In some embodiments in accordance with the various aspects of the present disclosure, the HER3-associated cancer comprises: (i) an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5; and/or (ii) an inactivating mutation to a gene selected from: PTEN, PPP2CA, PIK3R1, PIK3R2, NF1, BAD and PHLPP1.

In some embodiments, the HER3-associated cancer comprises one or more of: an activating mutation to KRAS, an activating mutation to PIK3CA, an activating mutation to BRAF, or an inactivating mutation to PTEN. In some embodiments, the HER3-associated cancer comprises one or more of: an activating mutation to KRAS, an activating mutation to PIK3CA, or an inactivating mutation to PTEN.

Also provided is a method of selecting a subject for treatment with an antigen-binding molecule which binds to HER3, comprising:
(a) analysing a subject's cancer in order to determine whether the cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation; and
(b) selecting a subject for treatment with an antigen-binding molecule which binds to HER3, where the subject's cancer is determined in step (a) not to comprise such a mutation.

In some embodiments, the method further comprises:
(c) administering an antigen-binding molecule which binds to HER3 to a subject selected for treatment in step (b).

Also provided is a method of selecting a subject for treatment with (i) an antagonist of HER3-mediated signalling and (ii) an antigen-binding molecule which binds to HER3, comprising:
(a) analysing a subject's cancer in order to determine whether the cancer comprises a mutation resulting in upregulation of HER3-mediated signalling; and
(b) selecting a subject for treatment with (i) an antagonist of HER3-mediated signalling and (ii) an antigen-binding molecule which binds to HER3, where the subject's cancer is determined in step (a) to comprise such a mutation.

In some embodiments, the method further comprises:
(c) administering (i) an antagonist of HER3-mediated signalling and (ii) an antigen-binding molecule which binds to HER3, to a subject selected for treatment in step (b).

In some embodiments in accordance with the various aspects of the present disclosure, the HER3-associated cancer is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck, lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

In some embodiments in accordance with the various aspects of the present disclosure, the antigen-binding molecule which binds to HER3 is selected from: 10D1F, seribantumab, elgemtumab, patritumab, GSK2849330, lumretuzumab, CDX-3379, AV-203, barecetamab, TK-A3, TK-A4, MP-EV20, 1A5-3D4, 9F7-F11, 16D3-C1, NG33, A5, F4, huHER3-8, REGN1400 and zenocutuzumab.

In some embodiments, the antigen-binding molecule which binds to HER3 comprises:
(i) a heavy chain variable (VH) region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48; and
(ii) a light chain variable (VL) region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74.

In some embodiments, the antigen-binding molecule comprises:
(i) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45; and
(ii) a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70.

In some embodiments, the antigen-binding molecule comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

In some embodiments, the antigen-binding molecule comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.

### DESCRIPTION

The present disclosure is based on the inventors' unexpected observation that cancers lacking mutation to genes encoding mediators of HER3-mediated signalling respond exceptionally well to treatment with anti-HER3 antibody. In particular, cancers having homozygous wildtype genotype for KRAS, PIK3CA and PTEN or a homozygous wildtype genotype for KRAS, PIK3CA, BRAF and PTEN were highly responsive to anti-HER3 antibody treatment.

### HER3 and HER3-Mediated Signalling

HER3 (also known e.g. as ERBB3, LCCS2, MDA-BF-1) is the protein identified by UniProt P21860. The structure and function of HER3 is described e.g. in Cho and Leahy Science (2002) 297 (5585):1330-1333, Singer et al., Journal of Biological Chemistry (2001) 276, 44266-44274, Roskoski et al., Pharmacol. Res. (2014) 79: 34-74, Bazley and Gullick Endocrine-Related Cancer (2005) S17-S27 and Mujoo et al., Oncotarget (2014) 5(21):10222-10236, each of which are hereby incorporated by reference in their entirety. HER3 is a single-pass transmembrane ErbB receptor tyrosine kinase having an N-terminal extracellular region (SEQ ID NO:9) comprising two leucine-rich subdomains (domains I and III, shown in SEQ ID NOs:15 and 17, respectively) and two cysteine-rich subdomains (domains II and IV, shown in SEQ ID NOs:16 and 18, respectively). Domain II comprises a β hairpin dimerisation loop (SEQ ID NO:19) which is involved in intermolecular interactions with other HER receptor molecules. The extracellular region is linked via a transmembrane region (SEQ ID NO:10) to a cytoplasmic region (SEQ ID NO:11).

The cytoplasmic region comprises a juxtamembrane segment (SEQ ID NO:12), a protein kinase domain (SEQ ID NO:13), and a C-terminal segment (SEQ ID NO:14).

In this specification “HER3” refers to HER3 from any species and includes HER3 isoforms, fragments, variants (including mutants) or homologues from any species.

As used herein, a “fragment”, “variant” or “homologue” of a protein may optionally be characterised as having at least 60%, preferably one of 70%, 75%, 80%, 85%, 90%, 91%, 92%, 93%, 94%, 95%, 96%, 97%, 98%, 99% or 100% amino acid sequence identity to the amino acid sequence of the reference protein (e.g. a reference isoform). In some embodiments, fragments, variants, isoforms and homologues of a reference protein may be characterised by ability to perform a function performed by the reference protein.

A “fragment” generally refers to a fraction of the reference protein. A “variant” generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An “isoform” generally refers to a variant of the reference protein expressed by the same species as the species of the reference protein (e.g. human HER3 isoforms 1 to 5 are all isoforms of one another). A “homologue” generally refers to a variant of the reference protein produced by a different species as compared to the species of the reference protein. For example, human HER3 isoform 1 (P21860-1, v1; SEQ ID NO:1) and Rhesus macaque HER3 (UniProt: F7HEH3-1, v2; SEQ ID NO:20) are homologues of one another. Homologues include orthologues.

A “fragment” of a reference protein may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein.

A fragment of HER3 may have a minimum length of one of 10, 20, 30, 40, 50, 100, 150, 200, 250, 300, 350, 400, 450, 500, 600, 700, 800, 900, 1000, 1100, 1200 amino acids, and may have a maximum length of one of 20, 30, 40, 50, 100, 150, 200, 250, 300, 350, 400, 450, 500, 600, 700, 800, 900, 1000, 1100, 1200, or 1300 amino acids.

In some embodiments, the HER3 is HER3 from a mammal (e.g. a primate (rhesus, cynomolgous, non-human primate or human) and/or a rodent (e.g. rat or murine) HER3). Isoforms, fragments, variants or homologues of HER3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of an immature or mature HER3 isoform from a given species, e.g. human.

Isoforms, fragments, variants or homologues may optionally be functional isoforms, fragments, variants or homologues, e.g. having a functional property/activity of the reference HER3 (e.g. human HER3 isoform 1), as determined by analysis by a suitable assay for the functional property/activity. For example, an isoform, fragment, variant or homologue of HER3 may display association with one or more of: HER2, NRG1 (type I, II, III, IV, V or VI) or NRG2 (a or p).

In some embodiments, the HER3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:1 to 8.

In some embodiments, a fragment of HER3 comprises, or consists of, an amino acid sequence having at least 70%, preferably one of 80%, 85%, 90%, 91%, 92%, 93%, 94%, 95%, 96%, 97%, 98%, 99% or 100% amino acid sequence identity to one of SEQ ID NOs:9 to 19, e.g. one of 9, 16 or 19.

Signalling through HER3 involves receptor heteromultimerization (i.e. with other ErBB receptors, e.g. HER2, EGFR) and consequent autophosphorylation by the protein kinase domain of tyrosine residues of the cytoplasmic region. HER3 lacks kinase activity and does not form stable homodimers. Therefore, HER3 must be transphosphorylated by binding to a kinase-active heterodimer partner (e.g. EGFR or HER2) for signal transduction to take place (Berger M B et al., FEBS Lett 2004; 569:332-6; Kim H H et al., Biochem J 1998; 334:189-95.).

Multimerization (e.g. dimerization) of HER receptor family members is required for activating cell growth signaling pathways, and HER3 can dimerize with other HER family members in both a ligand-dependent and ligand-independent manner. The HER3 extracellular domain (ECD) exists in a reversible equilibrium between a “closed” inactive conformation and an “open” active conformation, in which the dimerization arm within domain II is exposed to allow dimerization along the domain II dimerization interface, and in particular through the cysteine-rich CR1 region (Carraway, K. L., et al., Nature, 1997. 387(6632): 512-6; Riese, D. J., et al., Mol Cell Biol, 1995. 15(10): 5770-6; Harari, D., et al., Oncogene, 1999. 18(17): 2681-9; Zhang, D., et al., Proc Natl Acad Sci USA, 1997. 94(18): 9562-7; Meyer et al., Nature, 1995. 378(6555):386-90; Jura, N., et al., Proc Natl Acad Sci USA, 2009. 106(51): 21608-13; Fornaro, L., et al., Nat Rev Gastroenterol Hepatol, 2011. 8(7):369-83; Mota et al., Oncotarget (2015) 5:89284-306). HER3 is “activated” when the equilibrium is shifted in favor of the open conformation, increasing the probability of forming active heterodimers. The conventional model for activation is ligand-dependent, that is, the equilibrium shifts when HER3 in the open conformation is stabilized by binding of its ligand such as a neuregulin (NRG), e.g. NRG1 (also known as heregulin, HRG) or NRG2. In addition, the presence of any dimerization partner at a sufficient concentration will shift the equilibrium in favor of the open conformation, as they binds to and stabilize HER3 transiently in an open conformation. This is known as ligand-independent activation (Jura, N., et al., Proc Natl Acad Sci USA, 2009. 106(51): 21608-13; Fornaro, L., et al., Nat Rev Gastroenterol Hepatol, 2011. 8(7): p. 369-83; Mota et al., Oncotarget (2015) 5:89284-306).

Herein, “HER3-mediated signalling” refers to signalling mediated by HER3 and/or multimeric ErBB family member receptor complexes comprising HER3. “Signalling” refers to signal transduction and other cellular processes governing cellular activity. HER3-mediated signalling may be mediated by HER3 receptor-containing complexes, e.g. by heteromultimeric complexes comprising HER3 and other HER receptors (e.g. HER2, EGFR). HER3-mediated signalling may be ligand-dependent, e.g. triggered by binding of NRG (e.g. NRG1, NRG2), or may be ligand-independent.

HER3-mediated signalling progresses intracellularly through the MAPK/ERK and PI3K/AKT/mTOR pathways to promote cell survival and proliferation. HER3-mediated signalling is described e.g. in Gala and Chandarlapaty, Clin Cancer Res. (2014) 20(6): 1410-1416, Mishra et al., Oncol Rev. (2018) 12(1): 355, Baselga et al., Nat Rev Cancer (2009) 9:463-75, Yarden et al., Nat Rev Mol Cell Biol (2001) 2:35052073, Mota et al., Oncotarget (2015) 5:89284-306 and Haikala and Janne, Clin. Cancer Res. (2021) 27:3528-39, all of which are hereby incorporated by reference in their entirety.

Phosphorylated tyrosine residues in the protein kinase domains of HER3-containing receptor complexes recruit adaptor/effector proteins GRB2, via interaction with its SH2 domain. Upon ligand stimulation, the activated receptor (EGFR/HER2) undergoes autophosphorylation and provides phospho-tyrosine residues for recruiting GRB2. GRB2 binds via its SH3 domains to the guanine nucleotide exchange factor SOS. Activated SOS in GRB2-SOS complexes promotes removal of GDP from, and thereby activation of, Ras family GTPases such as H-Ras, N-Ras and K-Ras. Activated Ras GTPases in turn activate RAF kinases such as A-Raf, B-Raf and C-Raf. RAF kinases in turn phosphorylate and activate MEK1 and MEK2, which then phosphorylate and activate MAPKs (also known as ERKs). Activated MAPKs are able to directly regulate the activity of transcription factors such as c-Myc. Activated MAPKs also upregulate translation of mRNA into protein via phosphorylation of RSK, and consequent phosphorylation and activation of 40S ribosomal protein S6. Activated MAPKs also phosphorylate and activate MNK, which in turn phosphorylates and activates the transcription factor CREB.

Phosphorylated tyrosine residues in the protein kinase domain of HER3 also recruit the p85 subunit of PI3K, through its SH2 domain. Association of p85 causes allosteric activation of the lipid kinase p100α subunit of PI3K. Activated PI3K results in conversion of PIP2 to PIP3, which recruits AKT to be phosphorylated and activated by mTORC2 and PDK1. Phosphorylated AKT has a number of activities, including activating CREB and mTOR. PTEN antagonises signalling through the PI3K/AKT/mTOR pathway by dephosphorylating PIP3 to PIP2, and PP2A inhibits the PI3K/AKT/mTOR pathway by dephosphorylating AKT.

Oncogenic Src homology region 2 protein tyrosine phosphatase 2 (SHP2) promotes tumor progression and serves as a pivotal hub to connect multiple oncogenic signaling pathways, such as PI3K/Akt, Ras/Raf/MAPK (Dong et al., Front. Cell Dev. Biol., 11 Mar. 2021). GAB2 binds to GRB2 and becomes phosphorylated at multiple tyrosine residues, capable of binding to the SH2 domains of SHP2 and p85 (Adams et al., Mol Cancer Res. 2012 October; 10(10):1265-70; (Liu et al., Proc. Natl. Acad. Sci. U.S.A. (2016) 113, 984-989). The interactions induce conformation changes, relieving the auto-inhibition of the SHP2 catalytic site (Neel et al., Trends Biochem Sci. 2003 June; 28(6):284-93) and relieving the inhibition of p85 on the p110 catalytic subunit of PI3K (Cuevas et al., J Biol Chem. 2001 Jul. 20; 276(29):27455-6), respectively. SHP2 has been shown to activate RAS by direct dephosphorylation of RAS (Bunda et al., Nat Commun. 2015 Nov. 30; 60:8859), inhibition of RASGAP (RAS GTPase activating protein) (Neel et al., Trends Biochem Sci. 2003 June; 28(6):284-93) and SPRY (Hanafusa et al., J Biol Chem. 2004 May 28; 279(22):22992-5). SHP2 overexpression has been shown to enhance tumor invasion by activating the PI3K/Akt axis (Hu et al., Onco Targets Ther. (2017) 10, 3881-3891) while SHP2 knockdown inhibits cell migration and the tumor-promoting effect of SHP2 is partially related to Akt signaling (Cao et al., Pathol. Res. Pract. (2019) 215:152621).

STAT3 and 5 proteins are transcription factors that enhance the expression of p85a, p110a and AKT1 and thereby augment signaling through the PI3K/AKT signal transduction cascade (Radler et al., Mol Cell Endocrinol. 2017 Aug. 15; 451: 31-39). Upon activation by JAK2, phosphorylated STAT5 binds to the SH2 domain of the p85a regulatory subunit of PI3K in a PRL signaling-dependent manner suggesting that STAT5 may also directly participate in the signaling of PI3K complexes. Another kinase that phosphorylates EGFR is the cytokine-regulated tyrosine kinase Jak2, thus allowing MAPK activation even by a kinase-defective mutant of EGFR (Mishra et al., Oncol Rev. (2018) 12(1): 355, Baselga et al., Nat Rev Cancer (2009) 9:463-75). The collective observations in genetic models overexpressing or lacking active STAT5 and AKT or expressing mutant PTEN supports the notion that STAT5 functions as a survival factor during normal mammary gland development and as an oncogene during mammary carcinogenesis are mediated by the PI3K/AKT pathway (Radler et al., Mol Cell Endocrinol. 2017 Aug. 15; 451: 31-39).

### Cancers

The present disclosure relates to the treatment and prevention of cancers.

A cancer in accordance with the present disclosure may be any unwanted cell proliferation (or any disease manifesting itself by unwanted cell proliferation), neoplasm or tumor. The cancer may be benign or malignant. The cancer may be primary or secondary (e.g. metastatic). A neoplasm or tumor may be any abnormal growth or proliferation of cells, and may be located in (and/or derived from cells of) any organ/tissue.

A cancer may be of cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

A cancer may be, or may comprise, one or more tumors. A cancer may be a glioma, medulloblastoma, meningioma, neurofibroma, ependymoma, Schwannoma, neurofibrosarcoma, astrocytoma and oligodendroglioma, melanoma, mesothelioma, myeloma, lymphoma, Non-Hodgkin's lymphoma (NHL), Hodgkin's lymphoma, cutaneous T-cell lymphoma (CTCL), leukemia, chronic myelogenous leukemia (CML), acute myeloid leukemia (AML), chronic lymphocytic leukemia (CLL), myelodysplastic syndrome (MDS), hepatoma, epidermoid carcinoma, prostate cancer, breast cancer, lung cancer, NSCLC, colon cancer, ovarian cancer, pancreatic cancer, thymic cancer, hematologic cancer or sarcoma.

In some embodiments, a cancer according to the present disclosure is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck (SCCHN), lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

In some embodiments, a cancer according to the present disclosure is selected from: gastric cancer (e.g. gastric carcinoma, gastric adenocarcinoma, gastrointestinal adenocarcinoma), head and neck cancer (e.g. head and neck squamous cell carcinoma), breast cancer, ovarian cancer (e.g. ovarian carcinoma), lung cancer (e.g. NSCLC, lung adenocarcinoma, squamous lung cell carcinoma), melanoma, prostate cancer, oral cavity cancer (e.g. oropharyngeal cancer), renal cancer (e.g. renal cell carcinoma) or colorectal cancer (e.g. colorectal carcinoma), oesophageal cancer, pancreatic cancer, a solid cancer and a liquid cancer (i.e. a hematological cancer).

In some embodiments, the cancer comprises cells expressing an EGFR family member (e.g. HER3, EGFR, HER2 or HER4), and/or cells expressing a ligand for an EGFR family member. In some embodiments, the cancer to be treated/prevented is a cancer which is positive for an EGFR family member. In some embodiments, the cancer over-expresses an EGFR family member and/or a ligand for an EGFR family member. Overexpression of an EGFR family member and/or a ligand for an EGFR family member can be determined by detection of a level of expression by the cancer cells/tumor tissue which is greater than the level of expression by equivalent non-cancerous cells/non-tumor tissue.

Expression may be determined by any suitable means. Expression may be gene expression or protein expression. Gene expression can be determined e.g. by detection of mRNA encoding HER3, for example by quantitative real-time PCR (qRT-PCR). Protein expression can be determined e.g. by antibody-based methods, for example by western blot, immunohistochemistry, immunocytochemistry, flow cytometry, or ELISA.

In some embodiments, a cancer according to the present disclosure is a HER3-associated cancer.

HER3 and its association with and role in cancer is reviewed e.g. in Karachaliou et al., BioDrugs. (2017) 31(1):63-73 and Zhang et al., Acta Biochimica et Biophysica Sinica (2016) 48(1): 39-48, both of which are hereby incorporated by reference in their entirety.

As used herein, a “HER3-associated” cancer refers to a cancer for which gene and/or protein expression of HER3 is a risk factor for (e.g. is positively associated with), the onset, development, progression or severity of symptoms of the cancer. In some embodiments, the cancer over-expresses HER3. In some embodiments, the cancer may comprise cells having elevated gene and/or protein expression of HER3, e.g. relative to the level of expression by equivalent non-cancerous cells (e.g. non-cancerous cells of the same type, e.g. non-cancerous cells derived from the same organ/tissue).

In preferred embodiments, a HER3-associated cancer may be a cancer comprising cells expressing HER3. In some embodiments, the cancer comprises cells expressing HER3 protein. A cancer comprising cells expressing HER3 protein may be referred to as a “HER3-positive” cancer. In some embodiments, the cancer comprises cells expressing HER3 protein at the surface of the cell (i.e. in or at the cell membrane).

In some embodiments, a HER3-associated cancer may be selected from: ovarian cancer, breast cancer, prostate cancer, gastric cancer, bladder cancer, pancreatic cancer, lung cancer, melanoma, colorectal cancer, squamous cell carcinoma and oral cavity cancer.

Elevated plasma levels of NRG1 have been linked to de novo resistance to treatment with the anti-EGFR antibody cetuximab in colorectal cancer patients (Yonesaka et al. Transl Med. (2011) 3(99). Patients that responded to cetuximab treatment displayed significantly lower expression of NRG1 in tumor samples prior to treatment. Liu et al., Journal of Clinical Oncology (2016) 34 (36): 4345-4353 reports that in subjects having advanced, platinum-resistant or refractory ovarian cancer, NRG1-expressing cancers respond better to treatment with seribantumab.

In some embodiments, the cancer to be treated/prevented comprises cells expressing a ligand for HER3 (e.g. NRG1 and/or NRG2). In some embodiments, the cancer to be treated/prevented comprises cells expressing a level of expression of NRG1 and/or NRG2 which is greater than the level of expression by equivalent non-cancerous cells/non-tumor tissue. In some embodiments, the cancer comprises a mutation resulting in increased (gene and/or protein) expression of a ligand for HER3 (e.g. NRG1 and/or NRG2), relative to equivalent cells harbouring only the wildtype allele.

Mutations which cause increased expression of a ligand for HER3 are described e.g. in WO 2021/048274 A1, which is hereby incorporated by reference in its entirety. In some embodiments, a mutation resulting in increased expression of a ligand for HER3 is an NRG gene fusion. In some embodiments, the ligand for HER3 is the product of (i.e. a polypeptide encoded by) an NRG gene fusion. As used herein, an “NRG gene fusion” refers to a genetic variant encoding a polypeptide comprising (i) an amino acid sequence of an NRG protein (e.g. NRG1, NRG2, NRG3 or NRG4; e.g. NRG1 or NRG2), and (ii) an amino acid sequence of a protein other than the NRG protein.

NRG gene fusions are described e.g. in WO 2021/048274 A1 (incorporated by reference hereinabove). In some embodiments, an NRG gene fusion is selected from CLU-NRG1, CD74-NRG1, DOC4-NRG1, SLC3A2-NRG1, RBPMS-NRG1, WRN-NRG1, SDC4-NRG1, RAB21L1-NRG1, VAMP2-NRG1, KIF13B-NRG1, THAP7-NRG1, SMAD4-NRG1, MDK-NRG1, TNC-NRG1, DIP2B-NRG1, MRPL13-NRG1, PARP8-NRG1, ROCK1-NRG1, DPYSL2-NRG1, ATP1B1-NRG1, CDH6-NRG1, APP-NRG1, AKAPI3-NRG1, THBS1-NRG1, FOXA1-NRG1, PDE7A-NRG1, RAB31L1-NRG1, CDK1-NRG1, BMPRIB-NRG1, TNFRSF10B-NRG1, MCPH1-NRG1 and SLC12A2-NRG2.

Aspects and embodiments of the present disclosure are concerned with therapeutic and prophylactic intervention for the treatment/prevention of cancers characterised by the absence or presence of certain variants of genes encoding factors involved in HER3-mediated signalling.

### Mutations, Alleles and Genotypes

As used herein, a “mutation” refers to a difference relative to the most common nucleotide sequence of a given gene. A mutation may be or comprise insertion, deletion, substitution to, or larger-scale translocation/rearrangement of, the nucleotide sequence relative to the most common nucleotide sequence of the gene.

A mutation “resulting in” upregulation of signalling (e.g. HER3-mediated signalling, signalling through the MAPK/ERK pathway, signalling through the PI3K/AKT/mTOR pathway) may be a mutation which is known or predicted to cause an increase in the level of the relevant signalling in cells having one or more copies of an allele comprising the mutation, as compared to the level of the signalling by equivalent cells lacking a copy of an allele comprising the mutation (e.g. equivalent cells which are homozygous for the wildtype allele).

HER3-mediated signalling can be analysed e.g. using an assay of a correlate of HER3-mediated signalling, e.g. cell proliferation, and/or phosphorylation of one or more signal transduction molecules of the PI3K/AKT/mTOR and/or MAPK/ERK signal transduction pathways. For example, the level of PI3K/AKT/mTOR and/or MAPK/ERK signalling may be analysed by detection and quantification of the level of phosphorylation of one or more of the components of the PI3K/AKT/mTOR and/or MAPK/ERK pathways. Such analysis may be performed in vitro in a cell-based assay of HER3-mediated signalling, e.g. as described in Example 4.3 of 8.9 of WO 2019185878 A1.

The most common version of the nucleotide sequence of a given gene may be referred to as the wildtype allele of the gene. A version of the nucleotide sequence of a given gene comprising a mutation may be referred to as a mutant allele of the gene. It will be appreciated that the nucleotide sequence of a mutant allele of a given gene has a nucleotide sequence which is non-identical to the nucleotide sequence of the wildtype allele.

As used herein, an “activating mutation” refers to a mutation resulting in an increase in the level of expression of the relevant gene, and/or a mutation resulting in an increase in an activity of a product of the gene. Conversely, an “inactivating mutation” refers to a mutation resulting in a decrease in the level of expression of a given gene, and/or a mutation resulting in a decrease in an activity of a product of the gene.

In some embodiments, a mutation resulting in upregulation of HER3-mediated signalling is an activating mutation to a gene encoding a positive regulator of HER3-mediated signalling. In some embodiments, a mutation resulting in upregulation of HER3-mediated signalling is an inactivating mutation to a gene encoding a negative regulator of HER3-mediated signalling.

Herein, a cancer comprising cells having specified characteristics may be referred to herein simply as a cancer having those characteristics. It will be appreciated that in embodiments herein, cancers comprising cells having specified characteristics may be, or may comprise, one or more tumors comprising cells having those characteristics. That is, where a cancer is described as having a given mutation status, allele or genotype it will be appreciated that cells of the cancer have the relevant mutation status, allele or genotype. By way of illustration, where a cancer is described as comprising a given mutation, the cancer comprises cells comprising the mutation. Similarly, where a cancer is described as being homozygous for a given allele, the cancer comprises cells which are homozygous for the allele.

Where a cancer is described as having a given mutation status, allele or genotype, one or more cells of the cancer have the relevant mutation status, allele or genotype. In some embodiments, where a cancer is described as having a given mutation status, allele or genotype the majority (i.e. ≥50%) of cells of the cancer have the relevant mutation status, allele or genotype. In some embodiments, one of ≥60%, ≥65%, ≥70%, ≥75%, ≥80%, ≥85%, ≥90%, ≥95% or 100% of the cells of the cancer possess the relevant mutation status, allele or genotype.

In some embodiments, a cancer comprising a given mutation/allele/genotype may be a cancer in which ≥10% (e.g. one of ≥20%, ≥50%, ≥40%, ≥50%, ≥60%, ≥65%, ≥70%, ≥75%, ≥80%, ≥85%, ≥90%, ≥95% or 100%) of cells of the cancer comprise the mutation/allele/genotype. In some embodiments, a cancer not comprising a given mutation/allele/genotype may be a cancer in which <25% (e.g. one of ≤20%, ≤15%, ≤10%, ≤5%, ≤1% or none) of the cells of the cancer comprise the mutation/allele/genotype.

Herein, where a cell is described as comprising a given mutation, it will be appreciated that one or both alleles of the relevant gene(s) comprise such mutation (i.e. the cell is heterozygous or homozygous for the mutation/mutant allele). Conversely, where a cell is described as not comprising a given mutation, it will be appreciated that neither allele of the relevant gene(s) comprise such mutation (i.e. the cell is not homozygous or heterozygous for the mutation/mutant allele).

In some embodiments, an activating mutation according to the present disclosure may: increase transcription of the gene; increase the level of RNA encoded by the gene; decrease degradation of RNA encoded by the gene; increase the level of a protein encoded by the gene; increase (facilitate) normal splicing of pre-mRNA encoded by the gene; increase translation of mRNA encoding a protein encoded by the gene; increase (facilitate) normal post-translational processing of a protein encoded by the gene; increase (facilitate) normal trafficking of a protein encoded by the gene; decrease degradation of a protein encoded by the gene; increase the level of a function of a protein encoded by the gene; and/or confer a protein encoded by the gene with a novel property.

In some embodiments, an inactivating mutation according to the present disclosure may: decrease transcription of the gene; decrease the level of RNA encoded by the gene; increase degradation of RNA encoded by the gene; decrease the level of a protein encoded by the gene; decrease (disrupt) normal splicing of pre-mRNA encoded by the gene; decrease translation of mRNA encoding a protein encoded by the gene; decrease (disrupt) normal post-translational processing of a protein encoded by the gene; decrease (disrupt) normal trafficking of a protein encoded by the gene; increase degradation of a protein encoded by the gene; and/or decrease the level of a function of a protein encoded by the gene.

As used herein, a “positive regulator” of signalling through a given pathway refers to a factor whose expression/activity generally contributes positively to (i.e. potentiates, enhances) signalling through the relevant pathway. An increase in the level of expression and/or activity of a positive regulator may result in an increase in the level of signalling through the relevant pathway (e.g. as determined by analysis of a correlate of such signalling). A decrease in the level of expression and/or activity of a positive regulator may result in a decrease in the level of signalling through the relevant pathway.

A “negative regulator” of signalling through a given pathway refers to a factor whose expression/activity generally contributes negatively to (i.e. inhibits, antagonises) signalling through the relevant pathway. An increase in the level of expression and/or activity of a negative regulator may result in an decrease in the level of signalling through the relevant pathway (e.g. as determined by analysis of a correlate of such signalling). A decrease in the level of expression and/or activity of a negative regulator may result in an increase in the level of signalling through the relevant pathway.

Cancers not Homozygous for/Heterozygous for/Comprising Mutations Resulting in Upregulation of HER3-Mediated Signalling

Aspects and embodiments of the present disclosure relate to cancers which are not homozygous for, which are not heterozygous for, or which do not comprise one or more mutations resulting in upregulation of HER3-mediated signalling. Such cancers may be considered as being particularly sensitive/susceptible to (and therefore likely to respond well to) therapeutic/prophylactic intervention with an antigen-binding molecule which binds to HER3 (e.g. as a monotherapy).

In some aspects and embodiments according to the present disclosure, the cancer to be treated/prevented is not homozygous for a mutation resulting in upregulation of HER3-mediated signalling. In some embodiments, the cancer is not heterozygous for a mutation resulting in upregulation of HER3-mediated signalling. In some embodiments, the cancer does not comprise a mutation resulting in upregulation of HER3-mediated signalling.

In some embodiments, the cancer is not homozygous for an activating mutation to a gene encoding a positive regulator of HER3-mediated signalling. In some embodiments, the cancer is not heterozygous for an activating mutation to a gene encoding a positive regulator of HER3-mediated signalling. In some embodiments, the cancer does not comprise an activating mutation to a gene encoding a positive regulator of HER3-mediated signalling.

In some embodiments, the cancer is not homozygous for an inactivating mutation to a gene encoding a negative regulator of HER3-mediated signalling. In some embodiments, the cancer is not heterozygous for an inactivating mutation to a gene encoding a negative regulator of HER3-mediated signalling. In some embodiments, the cancer does not comprise an inactivating mutation to a gene encoding a negative regulator of HER3-mediated signalling.

In some embodiments, the cancer to be treated/prevented is not homozygous for a mutation resulting in upregulation of signalling through the MAPK/ERK pathway. In some embodiments, the cancer is not heterozygous for a mutation resulting in upregulation of signalling through the MAPK/ERK pathway. In some embodiments, the cancer does not comprise a mutation resulting in upregulation of signalling through the MAPK/ERK pathway.

In some embodiments, the cancer is not homozygous for an activating mutation to a gene encoding a positive regulator of signalling through the MAPK/ERK pathway. In some embodiments, the cancer is not heterozygous for an activating mutation to a gene encoding a positive regulator of signalling through the MAPK/ERK pathway. In some embodiments, the cancer does not comprise an activating mutation to a gene encoding a positive regulator of signalling through the MAPK/ERK pathway.

In some embodiments, the cancer is not homozygous for an inactivating mutation to a gene encoding a negative regulator of signalling through the MAPK/ERK pathway. In some embodiments, the cancer is not heterozygous for an inactivating mutation to a gene encoding a negative regulator of signalling through the MAPK/ERK pathway. In some embodiments, the cancer does not comprise an inactivating mutation to a gene encoding a negative regulator of signalling through the MAPK/ERK pathway.

In some embodiments, the cancer to be treated/prevented is not homozygous for a mutation resulting in upregulation of signalling through the PI3K/AKT/mTOR pathway. In some embodiments, the cancer is not heterozygous for a mutation resulting in upregulation of signalling through the PI3K/AKT/mTOR pathway. In some embodiments, the cancer does not comprise a mutation resulting in upregulation of signalling through the PI3K/AKT/mTOR pathway.

In some embodiments, the cancer is not homozygous for an activating mutation to a gene encoding a positive regulator of signalling through the PI3K/AKT/mTOR pathway. In some embodiments, the cancer is not heterozygous for an activating mutation to a gene encoding a positive regulator of signalling through the PI3K/AKT/mTOR pathway. In some embodiments, the cancer does not comprise an activating mutation to a gene encoding a positive regulator of signalling through the PI3K/AKT/mTOR pathway.

In some embodiments, the cancer is not homozygous for an inactivating mutation to a gene encoding a negative regulator of signalling through the PI3K/AKT/mTOR pathway. In some embodiments, the cancer is not heterozygous for an inactivating mutation to a gene encoding a negative regulator of signalling through the PI3K/AKT/mTOR pathway. In some embodiments, the cancer does not comprise an inactivating mutation to a gene encoding a negative regulator of signalling through the PI3K/AKT/mTOR pathway.

Herein, a positive regulator of HER3-mediated signalling/signalling through the MAPK/ERK pathway/signalling through the PI3K/AKT/mTOR pathway may be any factor that contributes positively to (i.e. potentiates) signalling through the given pathway, and/or a functional consequence thereof. A negative regulator of HER3-mediated signalling/signalling through the MAPK/ERK pathway/signalling through the PI3K/AKT/mTOR pathway may be any factor that contributes negatively to (i.e. antagonises, inhibits) signalling through the given pathway, and/or a functional consequence thereof.

In some embodiments, a gene encoding a positive regulator of HER3-mediated signalling may be selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5. In some embodiments, a gene encoding a positive regulator of HER3-mediated signalling may be selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF. In some embodiments, a gene encoding a positive regulator of HER3-mediated signalling may be selected from: KRAS, PIK3CA and PIK3CB. In some embodiments, a gene encoding a positive regulator of HER3-mediated signalling is KRAS. In some embodiments, a gene encoding a positive regulator of HER3-mediated signalling is PIK3CA. In some embodiments, a gene encoding a positive regulator of HER3-mediated signalling is PIK3CB.

In some embodiments, a gene encoding a positive regulator of signalling through the MAPK/ERK pathway may be selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1 and CREB1. In some embodiments, a gene encoding a positive regulator of signalling through the MAPK/ERK pathway may be selected from: ERBB3, ERBB2, ERBB4, EGFR, IGF1R, NRG1, NRG2, EGF, IRS2, GAB2, GRB2, PTPN11, SHP2, SOS1, HRAS, KRAS, NRAS, RAF1, MAP2K1, MAP2K2, MAPK1, MYC, RPS6KA1, RPS6, MKNK1, CREB1, and BRAF. In some embodiments, a gene encoding a positive regulator of signalling through the MAPK/ERK pathway is KRAS. In some embodiments, a gene encoding a positive regulator of signalling through the MAPK/ERK pathway is BRAF.

In some embodiments, a gene encoding a positive regulator of signalling through the PI3K/AKT/mTOR pathway may be selected from: ERBB3, ERBB2, ERBB4, EGFR, IGF1R, NRG1, NRG2, EGF, IRS2, GAB2, SHP2, CREB1, PIK3CA, PIK3CB, PIK3CD, MTOR, PDK1, AKT1, AKT2, AKT3, JAK2, STAT3 and STAT5. In some embodiments, a gene encoding a positive regulator of signalling through the PI3K/AKT/mTOR pathway is PIK3CA. In some embodiments, a gene encoding a positive regulator of signalling through the PI3K/AKT/mTOR pathway is PIK3CB.

In some embodiments, a gene encoding a negative regulator of HER3-mediated signalling may be selected from: PTEN, PPP2CA, PIK3R1, PIK3R2, NF1, BAD and PHLPP1. In some embodiments, a gene encoding a negative regulator of signalling through the PI3K/AKT/mTOR pathway may be selected from: PTEN, PPP2CA, PIK3R1, PIK3R2, BAD and PHLPP1. In some embodiments, a gene encoding a negative regulator of signalling through the MAPK/ERK pathway may be NF1. In some embodiments, a gene encoding a negative regulator of HER3-mediated signalling is PTEN. In some embodiments, a gene encoding a negative regulator of signalling through the PI3K/AKT/mTOR pathway is PTEN.

In some embodiments, the cancer does not comprise cells which are homozygous for an allele of a gene encoding a positive regulator of HER3-mediated signalling comprising an activating mutation. In some embodiments, the cancer does not comprise cells encoding an allele of a gene encoding a positive regulator of HER3-mediated signalling comprising an activating mutation. In some embodiments, in cells of the cancer, one or both copies of a gene encoding a positive regulator of HER3-mediated signalling is/are the wildtype allele. In some embodiments, the cancer does not comprise cells encoding a mutant allele of a gene encoding a positive regulator of HER3-mediated signalling. In some embodiments, the cancer does not comprise cells which are homozygous for an allele of a gene encoding a negative regulator of HER3-mediated signalling comprising an inactivating mutation. In some embodiments, the cancer does not comprise cells encoding an allele of a gene encoding a negative regulator of HER3-mediated signalling comprising an inactivating mutation. In some embodiments, in cells of the cancer, one or both copies of a gene encoding a negative regulator of HER3-mediated signalling is/are the wildtype allele. In some embodiments, the cancer does not comprise cells encoding a mutant allele of a gene encoding a negative regulator of HER3-mediated signalling.

In some embodiments, the cancer does not comprise cells which are homozygous for an allele of a gene encoding a positive regulator of signalling through the MAPK/ERK pathway comprising an activating mutation. In some embodiments, the cancer does not comprise cells encoding an allele of a gene encoding a positive regulator of signalling through the MAPK/ERK pathway comprising an activating mutation. In some embodiments, in cells of the cancer, one or both copies of a gene encoding a positive regulator of signalling through the MAPK/ERK pathway is/are the wildtype allele. In some embodiments, the cancer does not comprise cells encoding a mutant allele of a gene encoding a positive regulator of signalling through the MAPK/ERK pathway. In some embodiments, the cancer does not comprise cells which are homozygous for an allele of a gene encoding a negative regulator of signalling through the MAPK/ERK pathway comprising an inactivating mutation. In some embodiments, the cancer does not comprise cells encoding an allele of a gene encoding a negative regulator of signalling through the MAPK/ERK pathway comprising an inactivating mutation. In some embodiments, in cells of the cancer, one or both copies of a gene encoding a negative regulator of signalling through the MAPK/ERK pathway is/are the wildtype allele. In some embodiments, the cancer does not comprise cells encoding a mutant allele of a gene encoding a negative regulator of signalling through the MAPK/ERK pathway.

In some embodiments, the cancer does not comprise cells which are homozygous for an allele of a gene encoding a positive regulator of signalling through the PI3K/AKT/mTOR pathway comprising an activating mutation. In some embodiments, the cancer does not comprise cells encoding an allele of a gene encoding a positive regulator of signalling through the PI3K/AKT/mTOR pathway comprising an activating mutation. In some embodiments, in cells of the cancer, one or both copies of a gene encoding a positive regulator of signalling through the PI3K/AKT/mTOR pathway is/are the wildtype allele. In some embodiments, the cancer does not comprise cells encoding a mutant allele of a gene encoding a positive regulator of signalling through the PI3K/AKT/mTOR pathway. In some embodiments, the cancer does not comprise cells which are homozygous for an allele of a gene encoding a negative regulator of signalling through the PI3K/AKT/mTOR pathway comprising an inactivating mutation. In some embodiments, the cancer does not comprise cells encoding an allele of a gene encoding a negative regulator of signalling through the PI3K/AKT/mTOR pathway comprising an inactivating mutation. In some embodiments, in cells of the cancer, one or both copies of a gene encoding a negative regulator of signalling through the PI3K/AKT/mTOR pathway is/are the wildtype allele. In some embodiments, the cancer does not comprise cells encoding a mutant allele of a gene encoding a negative regulator of signalling through the PI3K/AKT/mTOR pathway.

In some embodiments, the cancer does not comprise cells which are homozygous for an allele of KRAS comprising an activating mutation. In some embodiments, the cancer does not comprise cells encoding an allele of KRAS comprising an activating mutation. In some embodiments, in cells of the cancer, one or both copies of KRAS is/are the wildtype allele. In some embodiments, the cancer does not comprise cells encoding a mutant allele of KRAS.

Activating mutations to KRAS are described e.g. in Hobbs and Der, Cancer Discov. (2019) 9(6):696-698, which is hereby incorporated by reference in its entirety. Activating mutations to KRAS include mutation to G12 (e.g. G12A, G12D, G12D, G12R, G12C, G12S and G12V), mutation to G13 (e.g. G13D, G13C), mutation to Q61 (e.g. Q61H, Q61L, Q61K, Q61R), mutation to A146 (e.g. A146T, A146V) and mutation to K117 (e.g. K117N). In some embodiments, an activating mutation to KRAS according to the present disclosure is G12C.

In some embodiments, the cancer does not comprise cells which are homozygous for an allele of PIK3CA comprising an activating mutation. In some embodiments, the cancer does not comprise cells encoding an allele of PIK3CA comprising an activating mutation. In some embodiments, in cells of the cancer, one or both copies of PIK3CA is/are the wildtype allele. In some embodiments, the cancer does not comprise cells encoding a mutant allele of PIK3CA.

Activating mutations to PIK3CA are described e.g. in Ligresti et al., Cell Cycle. (2009) 8(9): 1352-1358, which is hereby incorporated by reference in its entirety. Activating mutations to PIK3CA include mutation to H1047 (e.g. H1047R, H1047L), mutation to E542 (e.g. E542K, E542Q), mutation to E545 (e.g. E545K), mutation to P449 (e.g. P449T) and mutation to Q546 (e.g. Q546R). In some embodiments, an activating mutation to PIK3CA according to the present disclosure is Q546R or P449T.

In some embodiments, the cancer does not comprise cells which are homozygous for an allele of PIK3CB comprising an activating mutation. In some embodiments, the cancer does not comprise cells encoding an allele of PIK3CB comprising an activating mutation. In some embodiments, in cells of the cancer, one or both copies of PIK3CB is/are the wildtype allele. In some embodiments, the cancer does not comprise cells encoding a mutant allele of PIK3CB.

Activating mutations to PIK3CB are described e.g. in Nakanishi et al., Cancer Res. (2016) 76(5):1193-203, which is hereby incorporated by reference in its entirety. Activating mutations to PIK3CB include mutation to D1067 (e.g. D1067Y, D1067A, D1067V). In some embodiments, an activating mutation to PIK3CB according to the present disclosure is D1067Y.

In some embodiments, the cancer does not comprise cells which are homozygous for an allele of BRAF comprising an activating mutation. In some embodiments, the cancer does not comprise cells encoding an allele of BRAF comprising an activating mutation. In some embodiments, the cancer does not comprise cells encoding a mutant allele of BRAF.

Activating mutations to BRAF are described e.g. in Van Cutsem et al., Journal of Clinical Immunology (2011) 29(15): 2011-2019, which is hereby incorporated by reference in its entirety. Activating mutations to BRAF include mutations to V600 (e.g. V600E or V600K), T119 (e.g. T119S) and L597 (e.g. L597R). In some embodiments, an activating mutation to according to the present disclosure is V600E, V600K, T119S, or L597R.

In some embodiments, the cancer does not comprise cells which are homozygous for an allele of PTEN comprising an inactivating mutation. In some embodiments, the cancer does not comprise cells encoding an allele of PTEN comprising an inactivating mutation. In some embodiments, in cells of the cancer, one or both copies of PTEN is/are the wildtype allele. In some embodiments, the cancer does not comprise cells encoding a mutant allele of PTEN.

Inactivating mutations to PTEN are described e.g. in Chang et al., Biomolecules. (2019) 9(11):713, which is hereby incorporated by reference in its entirety. Inactivating mutations to PTEN include mutation to R130, mutation to R173, mutation to R233, mutation to K267, and mutation to N323.

In some embodiments, the cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

In some embodiments, the cancer: (i) comprises at least one gene encoding a positive regulator of HER3-mediated signalling that is not homozygous for an activating mutation; or (ii) comprises at least one gene encoding a negative regulator of HER3-mediated signalling that is not homozygous for an inactivating mutation.

In some embodiments, the cancer: (i) is not homozygous for an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5; or (ii) is not homozygous for an inactivating mutation to a gene selected from: PTEN, PPP2CA, PIK3R1, PIK3R2, NF1, BAD and PHLPP1.

In some embodiments, the cancer: (i) does not comprise an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5; or (ii) does not comprise an inactivating mutation to a gene selected from: PTEN, PPP2CA, PIK3R1, PIK3R2, NF1, BAD and PHLPP1.

In some embodiments, the cancer: (i) is not homozygous for an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF; or (ii) is not homozygous for an inactivating mutation to a gene selected from: PTEN, PPP2CA, PIK3R1, PIK3R2, NF1, BAD and PHLPP1.

In some embodiments, the cancer: (i) does not comprise an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF; or (ii) does not comprise an inactivating mutation to a gene selected from: PTEN, PPP2CA, PIK3R1, PIK3R2, NF1, BAD and PHLPP1.

Herein, it will be appreciated that reference to “a gene” means “at least one gene”, and includes “one or more genes”. Thus, “a gene” selected from a given list may be (depending on the number of genes recited in the list) 1 gene of the list, or may be one of 2, 3, 4, 5, 6, 7, 9, 10 or more, or all of the genes recited in the list. To be clear, a cancer satisfies the terms of the preceding paragraph provided: at least one of the genes recited in clause (i) lacks an activating mutation, or at least one of the genes recited in clause (ii) lacks an inactivating mutation.

In some embodiments, the cancer:
(i) is not homozygous for an activating mutation to KRAS and is not homozygous for an activating mutation to PIK3CA;
(ii) is not homozygous for an activating mutation to KRAS and is not homozygous for an inactivating mutation to PTEN;
(iii) is not homozygous for an activating mutation to KRAS and is not homozygous for an activating mutation to BRAF;
(iv) is not homozygous for an activating mutation to PIK3CA and is not homozygous for an inactivating mutation to PTEN;
(v) is not homozygous for an activating mutation to PIK3CA and is not homozygous for an activating mutation to BRAF;
(vi) is not homozygous for an inactivating mutation to PTEN and is not homozygous for an activating mutation to BRAF;
(vii) is not homozygous for an activating mutation to KRAS and is not homozygous for an activating mutation to PIK3CA and is not homozygous for an inactivating mutation to PTEN;
(viii) is not homozygous for an activating mutation to KRAS and is not homozygous for an activating mutation to PIK3CA and is not homozygous for an activating mutation to BRAF;
(ix) is not homozygous for an activating mutation to KRAS and is not homozygous for an inactivating mutation to PTEN and is not homozygous for an activating mutation to BRAF;
(x) is not homozygous for an activating mutation to PIK3CA and is not homozygous for an inactivating mutation to PTEN and is not homozygous for an activating mutation to BRAF; or
(xi) is not homozygous for an activating mutation to KRAS and is not homozygous for an activating mutation to PIK3CA and is not homozygous for an inactivating mutation to PTEN and is not homozygous for an activating mutation to BRAF.

In some embodiments, the cancer:
(i) does not comprise an activating mutation to KRAS and does not comprise an activating mutation to PIK3CA;
(ii) does not comprise an activating mutation to KRAS and does not comprise an inactivating mutation to PTEN;
(iii) does not comprise an activating mutation to KRAS and does not comprise an activating mutation to BRAF;
(iv) does not comprise an activating mutation to PIK3CA and does not comprise an inactivating mutation to PTEN;
(v) does not comprise an activating mutation to PIK3CA and does not comprise an activating mutation to BRAF;
(vi) does not comprise an inactivating mutation to PTEN and does not comprise an activating mutation to BRAF;
(vii) does not comprise an activating mutation to KRAS and does not comprise an activating mutation to PIK3CA and does not comprise an inactivating mutation to PTEN;
(viii) does not comprise an activating mutation to KRAS and does not comprise an activating mutation to PIK3CA and does not comprise an activating mutation to BRAF;
(ix) does not comprise an activating mutation to KRAS and does not comprise an inactivating mutation to PTEN and does not comprise an activating mutation to BRAF;
(x) does not comprise an activating mutation to PIK3CA and does not comprise an inactivating mutation to PTEN and does not comprise an activating mutation to BRAF;
(xi) does not comprise an activating mutation to KRAS and does not comprise an activating mutation to PIK3CA and does not comprise an inactivating mutation to PTEN and does not comprise an activating mutation to BRAF.
   Cancers Comprising/Heterozygous for/Homozygous for Mutations Resulting in Upregulation of HER3-Mediated Signalling

Aspects and embodiments of the present disclosure relate to cancers which comprise, are heterozygous for, or which are homozygous for one or more mutations resulting in upregulation of HER3-mediated signalling. Such cancers may be considered as being less sensitive to/more resistant to (and therefore less likely to respond well to) therapeutic/prophylactic intervention with an antigen-binding molecule which binds to HER3 (e.g. as a monotherapy).

In some aspects and embodiments according to the present disclosure—particularly aspects and embodiments concerning combination intervention using an inhibitor of HER3-mediated signalling and an antigen-binding molecule which binds to HER3—the cancer to be treated/prevented does comprise a mutation resulting in upregulation of HER3-mediated signalling. In some embodiments, the cancer is heterozygous for a mutation resulting in upregulation of HER3-mediated signalling. In some embodiments, the cancer is homozygous for a mutation resulting in upregulation of HER3-mediated signalling.

In some embodiments, the cancer comprises an activating mutation to a gene encoding a positive regulator of HER3-mediated signalling. In some embodiments, the cancer is heterozygous for an activating mutation to a gene encoding a positive regulator of HER3-mediated signalling. In some embodiments, the cancer is homozygous for an activating mutation to a gene encoding a positive regulator of HER3-mediated signalling.

In some embodiments, the cancer comprises an inactivating mutation to a gene encoding a negative regulator of HER3-mediated signalling. In some embodiments, the cancer is heterozygous for an inactivating mutation to a gene encoding a negative regulator of HER3-mediated signalling. In some embodiments, the cancer is homozygous for an inactivating mutation to a gene encoding a negative regulator of HER3-mediated signalling.

In some embodiments, the cancer comprises a mutation resulting in upregulation of signalling through the MAPK/ERK pathway. In some embodiments, the cancer is heterozygous for a mutation resulting in upregulation of signalling through the MAPK/ERK pathway. In some embodiments, the cancer is homozygous for a mutation resulting in upregulation of signalling through the MAPK/ERK pathway.

In some embodiments, the cancer comprises an activating mutation to a gene encoding a positive regulator of signalling through the MAPK/ERK pathway. In some embodiments, the cancer is heterozygous for an activating mutation to a gene encoding a positive regulator of signalling through the MAPK/ERK pathway. In some embodiments, the cancer is homozygous for an activating mutation to a gene encoding a positive regulator of signalling through the MAPK/ERK pathway.

In some embodiments, the cancer comprises an inactivating mutation to a gene encoding a negative regulator of signalling through the MAPK/ERK pathway. In some embodiments, the cancer is heterozygous for an inactivating mutation to a gene encoding a negative regulator of signalling through the MAPK/ERK pathway. In some embodiments, the cancer is homozygous for an inactivating mutation to a gene encoding a negative regulator of signalling through the MAPK/ERK pathway.

In some embodiments, the cancer comprises a mutation resulting in upregulation of signalling through the PI3K/AKT/mTOR pathway. In some embodiments, the cancer is heterozygous for a mutation resulting in upregulation of signalling through the PI3K/AKT/mTOR pathway. In some embodiments, the cancer is homozygous for a mutation resulting in upregulation of signalling through the PI3K/AKT/mTOR pathway.

In some embodiments, the cancer comprises an activating mutation to a gene encoding a positive regulator of signalling through the PI3K/AKT/mTOR pathway. In some embodiments, the cancer is heterozygous for an activating mutation to a gene encoding a positive regulator of signalling through the PI3K/AKT/mTOR pathway. In some embodiments, the cancer is homozygous for an activating mutation to a gene encoding a positive regulator of signalling through the PI3K/AKT/mTOR pathway.

In some embodiments, the cancer comprises an inactivating mutation to a gene encoding a negative regulator of signalling through the PI3K/AKT/mTOR pathway. In some embodiments, the cancer is heterozygous for an inactivating mutation to a gene encoding a negative regulator of signalling through the PI3K/AKT/mTOR pathway. In some embodiments, the cancer is homozygous for an inactivating mutation to a gene encoding a negative regulator of signalling through the PI3K/AKT/mTOR pathway.

In some embodiments, the cancer comprises cells encoding a mutant allele of a gene encoding a negative regulator of HER3-mediated signalling. In some embodiments, in cells of the cancer, one or both copies of a gene encoding a negative regulator of HER3-mediated signalling is/are not the wildtype allele. In some embodiments, the cancer comprises cells encoding an allele of a gene encoding a negative regulator of HER3-mediated signalling comprising an inactivating mutation. In some embodiments, the cancer comprises cells which are homozygous for an allele of a gene encoding a negative regulator of HER3-mediated signalling comprising an inactivating mutation. In some embodiments, the cancer comprises cells encoding a mutant allele of a gene encoding a positive regulator of HER3-mediated signalling. In some embodiments, in cells of the cancer, one or both copies of a gene encoding a positive regulator of HER3-mediated signalling is/are not the wildtype allele. In some embodiments, the cancer comprises cells encoding an allele of a gene encoding a positive regulator of HER3-mediated signalling comprising an activating mutation. In some embodiments, the cancer comprises cells which are homozygous for an allele of a gene encoding a positive regulator of HER3-mediated signalling comprising an activating mutation.

In some embodiments, the cancer comprises cells encoding a mutant allele of a gene encoding a negative regulator of signalling through the MAPK/ERK pathway. In some embodiments, in cells of the cancer, one or both copies of a gene encoding a negative regulator of signalling through the MAPK/ERK pathway is/are not the wildtype allele. In some embodiments, the cancer comprises cells encoding an allele of a gene encoding a negative regulator of signalling through the MAPK/ERK pathway comprising an inactivating mutation. In some embodiments, the cancer comprises cells which are homozygous for an allele of a gene encoding a negative regulator of signalling through the MAPK/ERK pathway comprising an inactivating mutation. In some embodiments, the cancer comprises cells encoding a mutant allele of a gene encoding a positive regulator of signalling through the MAPK/ERK pathway. In some embodiments, in cells of the cancer, one or both copies of a gene encoding a positive regulator of signalling through the MAPK/ERK pathway is/are not the wildtype allele. In some embodiments, the cancer comprises cells encoding an allele of a gene encoding a positive regulator of signalling through the MAPK/ERK pathway comprising an activating mutation. In some embodiments, the cancer comprises cells which are homozygous for an allele of a gene encoding a positive regulator of signalling through the MAPK/ERK pathway comprising an activating mutation.

In some embodiments, the cancer comprises cells encoding a mutant allele of a gene encoding a negative regulator of signalling through the PI3K/AKT/mTOR pathway. In some embodiments, in cells of the cancer, one or both copies of a gene encoding a negative regulator of signalling through the PI3K/AKT/mTOR pathway is/are not the wildtype allele. In some embodiments, the cancer comprises cells encoding an allele of a gene encoding a negative regulator of signalling through the PI3K/AKT/mTOR pathway comprising an inactivating mutation. In some embodiments, the cancer comprises cells which are homozygous for an allele of a gene encoding a negative regulator of signalling through the PI3K/AKT/mTOR pathway comprising an inactivating mutation. In some embodiments, the cancer comprises cells encoding a mutant allele of a gene encoding a positive regulator of signalling through the PI3K/AKT/mTOR pathway. In some embodiments, in cells of the cancer, one or both copies of a gene encoding a positive regulator of signalling through the PI3K/AKT/mTOR pathway is/are not the wildtype allele. In some embodiments, the cancer comprises cells encoding an allele of a gene encoding a positive regulator of signalling through the PI3K/AKT/mTOR pathway comprising an activating mutation. In some embodiments, the cancer comprises cells which are homozygous for an allele of a gene encoding a positive regulator of signalling through the PI3K/AKT/mTOR pathway comprising an activating mutation.

In some embodiments, the cancer comprises cells encoding a mutant allele of KRAS. In some embodiments, in cells of the cancer, one or both copies of KRAS is/are not the wildtype allele. In some embodiments, the cancer comprises cells encoding an allele of KRAS comprising an activating mutation.

In some embodiments, the cancer comprises cells which are homozygous for an allele of KRAS comprising an activating mutation.

In some embodiments, the cancer comprises cells encoding a mutant allele of PIK3CA. In some embodiments, in cells of the cancer, one or both copies of PIK3CA is/are not the wildtype allele. In some embodiments, the cancer comprises cells encoding an allele of PIK3CA comprising an activating mutation. In some embodiments, the cancer comprises cells which are homozygous for an allele of PIK3CA comprising an activating mutation.

In some embodiments, the cancer comprises cells encoding a mutant allele of PIK3CB. In some embodiments, in cells of the cancer, one or both copies of PIK3CB is/are not the wildtype allele. In some embodiments, the cancer comprises cells encoding an allele of PIK3CB comprising an activating mutation. In some embodiments, the cancer comprises cells which are homozygous for an allele of PIK3CB comprising an activating mutation.

In some embodiments, the cancer comprises cells encoding a mutant allele of BRAF. In some embodiments, in cells of the cancer, one or both copies of BRAF is/are not the wildtype allele. In some embodiments, the cancer comprises cells encoding an allele of BRAF comprising an activating mutation.

In some embodiments, the cancer comprises cells which are homozygous for an allele of BRAF comprising an activating mutation.

In some embodiments, the cancer comprises cells encoding a mutant allele of PTEN. In some embodiments, in cells of the cancer, one or both copies of PTEN is/are not the wildtype allele. In some embodiments, the cancer comprises cells encoding an allele of PTEN comprising an inactivating mutation. In some embodiments, the cancer comprises cells which are homozygous for an allele of PTEN comprising an inactivating mutation.

In some embodiments, the cancer: (i) comprises at least one gene encoding a positive regulator of HER3-mediated signalling that comprises an activating mutation; or (ii) comprises at least one gene encoding a negative regulator of HER3-mediated signalling that comprises an inactivating mutation.

In some embodiments, the cancer: (i) comprises at least one gene encoding a positive regulator of HER3-mediated signalling that is homozygous for an activating mutation; or (ii) comprises at least one gene encoding a negative regulator of HER3-mediated signalling that is homozygous for an inactivating mutation.

In some embodiments, the cancer: (i) comprises an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5; or (ii) comprises an inactivating mutation to a gene selected from: PTEN, PPP2CA, PIK3R1, PIK3R2, NF1, BAD and PHLPP1. In some embodiments, the cancer: (i) comprises an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF; or (ii) comprises an inactivating mutation to a gene selected from: PTEN, PPP2CA, PIK3R1, PIK3R2, NF1, BAD and PHLPP1.

To be clear, a cancer satisfies the terms of the preceding paragraph provided: at least one of the genes recited in clause (i) comprises an activating mutation, or at least one of the genes recited in clause (ii) comprises an inactivating mutation.

In some embodiments, the cancer: (i) is homozygous for an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3 and STAT5; or (ii) is homozygous for an inactivating mutation to a gene selected from: PTEN, PPP2CA, PIK3R1, PIK3R2, NF1, BAD and PHLPP1. In some embodiments, the cancer: (i) is homozygous for an activating mutation to a gene selected from: KRAS, PIK3CA, PIK3CB, PIK3CD, ERBB3, ERBB2, ERBB4, EGFR, IGF1R, NRG1, NRG2, EGF, IRS2,GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF; or (ii) is homozygous for an inactivating mutation to a gene selected from: PTEN, PPP2CA, PIK3R1, PIK3R2, NF1, BAD and PHLPP1.

In some embodiments, the cancer:
(i) comprises an activating mutation to KRAS and/or comprises an activating mutation to PIK3CA;
(ii) comprises an activating mutation to KRAS and/or comprises an inactivating mutation to PTEN;
(iii) comprises an activating mutation to KRAS and/or comprises an activating mutation to BRAF;
(iv) comprises an activating mutation to PIK3CA and/or comprises an inactivating mutation to PTEN;
(v) comprises an activating mutation to PIK3CA and/or comprises an activating mutation to BRAF;
(vi) comprises an inactivating mutation to PTEN and/or comprises an activating mutation to BRAF;
(vii) comprises an activating mutation to KRAS and/or comprises an activating mutation to PIK3CA and/or comprises an inactivating mutation to PTEN;
(viii) comprises an activating mutation to KRAS and/or comprises an activating mutation to PIK3CA and/or comprises an activating mutation to BRAF;
(ix) comprises an activating mutation to KRAS and/or comprises an inactivating mutation to PTEN and/or comprises an activating mutation to BRAF;
(x) comprises an activating mutation to PIK3CA and/or comprises an inactivating mutation to PTEN and/or comprises an activating mutation to BRAF; or
(xi) comprises an activating mutation to KRAS and/or comprises an activating mutation to PIK3CA and/or comprises an inactivating mutation to PTEN and/or comprises an activating mutation to BRAF.

In some embodiments, the cancer:
(i) is homozygous for an activating mutation to KRAS and/or is homozygous for an activating mutation to PIK3CA;
(ii) is homozygous for an activating mutation to KRAS and/or is homozygous for an inactivating mutation to PTEN;
(iii) is homozygous for an activating mutation to KRAS and/or is homozygous for an activating mutation to BRAF;
(iv) is homozygous for an activating mutation to PIK3CA and/or is homozygous for an inactivating mutation to PTEN;
(v) is homozygous for an activating mutation to PIK3CA and/or is homozygous for an activating mutation to BRAF;
(vi) is homozygous for an inactivating mutation to PTEN and/or is homozygous for an activating mutation to BRAF;
(vii) is homozygous for an activating mutation to KRAS and/or is homozygous for an activating mutation to PIK3CA and/or is homozygous for an inactivating mutation to PTEN;
(viii) is homozygous for an activating mutation to KRAS and/or is homozygous for an activating mutation to PIK3CA and/or is homozygous for an activating mutation to BRAF;
(ix) is homozygous for an activating mutation to KRAS and/or is homozygous for an inactivating mutation to PTEN and/or is homozygous for an activating mutation to BRAF;
(x) is homozygous for an activating mutation to PIK3CA and/or is homozygous for an inactivating mutation to PTEN and/or is homozygous for an activating mutation to BRAF; or
(xi) is homozygous for an activating mutation to KRAS and/or is homozygous for an activating mutation to PIK3CA and/or is homozygous for an inactivating mutation to PTEN and/or is homozygous for an activating mutation to BRAF.

### Antigen-Binding Molecules

The present disclosure relates to the therapeutic and prophylactic use of antigen-binding molecules which bind to HER3.

An “antigen-binding molecule” refers to a molecule which is capable of binding to a target antigen. Antigen-binding molecules include e.g. monoclonal antibodies, polyclonal antibodies, monospecific and multispecific antibodies (e.g., bispecific antibodies), and antibody fragments (e.g. Fv, scFv, Fab, scFab, F(ab′)₂, Fab₂, diabodies, triabodies, scFv-Fc, minibodies, single domain antibodies (e.g. VhH), etc.), as long as they display binding to the relevant target molecule(s).

Antigen-binding molecules according to the present disclosure also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody. For example, antigen-binding molecules according to the present disclosure include antibody-drug conjugates (ADCs) comprising a (cytotoxic) drug moiety (e.g. as described hereinbelow). Antigen-binding molecules according to the present disclosure also include multispecific antigen-binding molecules such as immune cell engager molecules comprising a domain for recruiting (effector) immune cells (reviewed e.g. in Goebeler and Bargou, Nat. Rev. Clin. Oncol. (2020) 17: 418-434 and Ellerman, Methods (2019) 154:102-117, both of which are hereby incorporated by reference in their entirety), including BiTEs, BiKEs and TriKEs. Antigen-binding molecules according to the present disclosure also include chimeric antigen receptors (CARs), which are recombinant receptors providing both antigen-binding and T cell activating functions (CAR structure, function and engineering is reviewed e.g. in Dotti et al., Immunol Rev (2014) 257(1), which is hereby incorporated by reference in its entirety).

The antigen-binding molecule of the present disclosure comprises a moiety capable of binding to a target antigen(s). In some embodiments, the moiety capable of binding to a target antigen comprises an antibody heavy chain variable region (VH) and an antibody light chain variable region (VL) of an antibody capable of specific binding to the target antigen. In some embodiments, the moiety capable of binding to a target antigen comprises or consists of an aptamer capable of binding to the target antigen, e.g. a nucleic acid aptamer (reviewed, for example, in Zhou and Rossi Nat Rev Drug Discov. 2017 16(3):181-202). In some embodiments, the moiety capable of binding to a target antigen comprises or consists of a antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)) affilin, armadillo repeat protein (ArmRP), OBody or fibronectin—reviewed e.g. in Reverdatto et al., Curr Top Med Chem. 2015; 15(12): 1082-1101, which is hereby incorporated by reference in its entirety (see also e.g. Boersma et al., J Biol Chem (2011) 286:41273-85 and Emanuel et al., Mabs (2011) 3:38-48).

The antigen-binding molecules of the present disclosure generally comprise an antigen-binding domain comprising a VH and a VL of an antibody capable of specific binding to the target antigen. The antigen-binding domain formed by a VH and a VL may also be referred to herein as an Fv region.

An antigen-binding molecule may be, or may comprise, an antigen-binding polypeptide, or an antigen-binding polypeptide complex. An antigen-binding molecule may comprise more than one polypeptide which together form an antigen-binding domain. The polypeptides may associate covalently or non-covalently. In some embodiments the polypeptides form part of a larger polypeptide comprising the polypeptides (e.g. in the case of scFv comprising VH and VL, or in the case of scFab comprising VH-CH1 and VL-CL).

An antigen-binding molecule may refer to a non-covalent or covalent complex of more than one polypeptide (e.g. 2, 3, 4, 6, or 8 polypeptides), e.g. an IgG-like antigen-binding molecule comprising two heavy chain polypeptides and two light chain polypeptides.

The antigen-binding molecules of the present disclosure may be designed and prepared using the sequences of monoclonal antibodies (mAbs) capable of binding to HER3. Antigen-binding regions of antibodies, such as single chain variable fragment (scFv), Fab and F(ab′)2 fragments may also be used/provided. An “antigen-binding region” is any fragment of an antibody which is capable of binding to the target for which the given antibody is specific.

Antibodies generally comprise six complementarity-determining regions (CDRs); three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody which binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue):D413-22), which uses the IMGT V-DOMAIN numbering rules as described in Lefranc et al., Dev. Comp. Immunol. (2003) 27:55-77.

In some embodiments, the antigen-binding molecule comprises the CDRs of an antigen-binding molecule which is capable of binding to HER3. In some embodiments, the antigen-binding molecule comprises the FRs of an antigen-binding molecule which is capable of binding to HER3. In some embodiments, the antigen-binding molecule comprises the CDRs and the FRs of an antigen-binding molecule which is capable of binding to HER3. That is, in some embodiments the antigen-binding molecule comprises the VH region and the VL region of an antigen-binding molecule which is capable of binding to HER3.

In some embodiments, an antigen-binding molecule which is capable of binding to HER3 according to the present disclosure may be selected from: any embodiment of an antigen-binding molecule described in WO 2019185878 A1 (which is hereby incorporated by reference in its entirety), 10D1F (described e.g. in WO 2019185878 A1), seribantumab (also known as MM-121, described e.g. in Schoeberl et al., Sci. Signal. (2009) 2(77): ra31), elgemtumab (also known as LJM-716, described e.g. in Garner et al., Cancer Res (2013) 73: 6024-6035), patritumab (also known as U-1287 and AMG-888, described e.g. in Shimizu et al., Cancer Chemother Pharmacol. (2017) 79(3):489-495), GSK2849330 (described e.g. in Clarke et al., Eur J Cancer. (2014) 50:98-9), lumretuzumab (also known as RG7116 and RO-5479599, described e.g. in Mirschberger et al., Cancer Research (2013) 73(16) 5183-5194), CDX-3379 (also known as KTN3379, described e.g. in Lee et al., Proc Natl Acad Sci USA. 2015 Oct. 27; 112(43):13225), AV-203 (also known as CAN-017, described e.g. in Meetze et al., Eur J Cancer 2012; 48:126), barecetamab (also known as ISU104, described e.g. in Kim et al., Cancer Res (2018) 78(13 Suppl): Abstract #830), TK-A3, TK-A4 (described e.g. in Malm et al., MAbs (2016) 8:1195-209), MP-EV20 (described e.g. in Sala et al., Transl. Oncol. (2013) 6:676-84), 1A5-3D4 (described e.g. in Wang et al., Cancer Lett (2016) 380:20-30), 9F7-F11, 16D3-C1 (described e.g. in Lazrek et al., Neoplasia (2013) 15:335-47), NG33, A5, F4 (described e.g. in Gaborit et al., PNAS USA (2015) 112:839-44), huHER3-8 (described e.g. in Kugel et al., Cancer Res. (2014) 74:4122-32), REGN1400 (described e.g. in Zhang et al., Mol Cancer Ther (2014) 13:1345-1355) and zenocutuzumab (also known as MCLA-128, described e.g. in de Vries Schultink et al., Clin Pharmacokinet. (2020) 59: 875-884).

In some embodiments, the antigen-binding molecule is selected from 10D1F and seribantumab. In some embodiments, the antigen-binding molecule is 10D1F.

In some embodiments, the antigen-binding molecule comprises the CDRs of, or comprises the VH and VL of, a HER3-binding antibody clone selected from 10D1_c89, 10D1, 10D1_c75, 10D1_c76, 10D1_c77, 10D1_c78v1, 10D1_c78v2, 10D1_11B, 10D1_c85v1, 10D1_c85v2, 10D1_c85o1, 10D1_c85o2, 10D1_c87, 10D1_c90, 10D1_c91, 10D1_c92 and 10D1_c93.

In some embodiments, the antigen-binding molecule comprises:
(1) a VH region incorporating the following CDRs:
   HC-CDR1 having the amino acid sequence of SEQ ID NO:40
   HC-CDR2 having the amino acid sequence of SEQ ID NO:43
   HC-CDR3 having the amino acid sequence of SEQ ID NO:48,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:66
   LC-CDR2 having the amino acid sequence of SEQ ID NO:69
   LC-CDR3 having the amino acid sequence of SEQ ID NO:74;
   or a variant thereof in which one or two or three amino acids in one or more of LC-CDR1, LC-CDR2 or LC-CDR3 are substituted with another amino acid.
(2) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(3) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:64
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(4) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:65
   LC-CDR2 having the amino acid sequence of SEQ ID NO:67
   LC-CDR3 having the amino acid sequence of SEQ ID NO:71;
   or a variant thereof in which one or two or three amino acids in one or more of LC-CDR1, LC-CDR2 or LC-CDR3 are substituted with another amino acid.
(5) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(6) a VH region incorporating the following CDRs:
   HC-CDR1 having the amino acid sequence of SEQ ID NO:39
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(7) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(8) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:46,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(9) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:47,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:68
   LC-CDR3 having the amino acid sequence of SEQ ID NO:70;
   or a variant thereof in which one or two or three amino acids in one or more of LC-CDR1, LC-CDR2 or LC-CDR3 are substituted with another amino acid.
(10) a VH region incorporating the following CDRs:
   HC-CDR1 having the amino acid sequence of SEQ ID NO:38
   HC-CDR2 having the amino acid sequence of SEQ ID NO:42
   HC-CDR3 having the amino acid sequence of SEQ ID NO:45,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:72;
   or a variant thereof in which one or two or three amino acids in one or more of LC-CDR1, LC-CDR2 or LC-CDR3 are substituted with another amino acid.
(11) a VH region incorporating the following CDRs:
   HC-CDR1 having the amino acid sequence of SEQ ID NO:38
   HC-CDR2 having the amino acid sequence of SEQ ID NO:41
   HC-CDR3 having the amino acid sequence of SEQ ID NO:44,
   or a variant thereof in which one or two or three amino acids in one or more of HC-CDR1, HC-CDR2, or HC-CDR3 are substituted with another amino acid; and
a VL region incorporating the following CDRs:
   LC-CDR1 having the amino acid sequence of SEQ ID NO:63
   LC-CDR2 having the amino acid sequence of SEQ ID NO:67
   LC-CDR3 having the amino acid sequence of SEQ ID NO:73;
   or a variant thereof in which one or two or three amino acids in one or more of LC-CDR1, LC-CDR2 or LC-CDR3 are substituted with another amino acid.

In some embodiments, the antigen-binding molecule comprises:
(12) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:21; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:49.
(13) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:50.
(14) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:51.
(15) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.
(16) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(17) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(18) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.
(19) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:28; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(20) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:29; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(21) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(22) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54.
(23) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:57.
(24) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:33; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:58.
(25) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:34; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:59.
(26) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:35; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:60.
(27) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:61.
(28) a VH region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:32; and a VL region comprising an amino acid sequence having at least 70% sequence identity, more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:62.

In embodiments in accordance with the present disclosure in which one or more amino acids are substituted with another amino acid, the substitutions may be conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g. target binding) of the antigen-binding molecule comprising the substitution as compared to the equivalent unsubstituted molecule.

The VH and VL region of an antigen-binding region of an antibody together constitute the Fv region. In some embodiments, the antigen-binding molecule according to the present disclosure comprises, or consists of, an Fv region which binds to HER3. In some embodiments the VH and VL regions of the Fv are provided as single polypeptide joined by a linker region, i.e. a single chain Fv (scFv).

In some embodiments, the antigen-binding molecule of the present disclosure comprises one or more regions of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM.

The VL and light chain constant (CL) region, and the VH region and heavy chain constant 1 (CH1) region of an antigen-binding region of an antibody together constitute the Fab region. In some embodiments, the antigen-binding molecule of the present disclosure comprises, or consists of, a Fab region which binds to HER3.

In some embodiments, the antigen-binding molecule described herein comprises, or consists of, a whole antibody which binds to HER3. As used herein, “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202): S41-S52, which is hereby incorporated by reference in its entirety.

Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments, the antigen-binding molecule comprises, or consists of, an IgG (e.g. IgG1, IgG2, IgG3, IgG4), IgA (e.g. IgA1, IgA2), IgD, IgE, or IgM which binds to HER3.

In some embodiments, the antigen-binding molecule comprises, or consists of:
(i) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:75; and
(ii) one or more (e.g. two) polypeptides comprising, or consisting of, an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:76.

Antigen-binding molecules according to the present disclosure may be provided in the form of compositions comprising such antigen-binding molecules. The antigen-binding molecules, may be formulated as pharmaceutical compositions or medicaments for clinical use and may comprise a pharmaceutically acceptable carrier, diluent, excipient or adjuvant. Compositions may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration, which may include injection or infusion.

Suitable formulations may comprise the antigen-binding molecule in a sterile or isotonic medium. Medicaments and pharmaceutical compositions may be formulated in fluid, including gel, form. Fluid formulations may be formulated for administration by injection or infusion (e.g. via cannula) to the blood, a tumor, or a selected region of the human or animal body. In some embodiments, compositions may be formulated for injection or infusion, e.g. into a blood vessel or tumor.

### Antagonists of HER3-Mediated Signalling

Aspects and embodiments of the present disclosure relate to therapeutic/prophylactic intervention with (i) an antagonist of HER3-mediated signalling, and (ii) an antigen-binding molecule which binds to HER3.

While HER3-binding antigen-binding molecules according to the present disclosure may behave as antagonists of HER3-mediated signalling, it will be appreciated that in accordance with aspects and embodiments of the present disclosure relating to combination treatment, components (i) and (ii) of the combination are preferably non-identical (i.e. they are different agents).

Treatment with an antagonist of HER3-mediated signalling in accordance with the present disclosure is contemplated, in particular, in connection with therapeutic/prophylactic intervention for the treatment/prevention of cancers described in the section entitled “cancers comprising/heterozygous for/homozygous for mutations resulting in upregulation of HER3-mediated signalling” hereinabove. In accordance with such embodiments, treatment with an antagonist of HER3-mediated signalling may be effective to restore the level of signalling to the level observed in the absence of mutation(s) (i.e. the level of signalling by equivalent cells harbouring only the wildtype allele(s)).

In this way, treatment with an antagonist of HER3-mediated signalling may be useful to condition a subject for treatment with a HER3-binding antigen-binding molecule described herein. That is, administration of an antagonist of HER3-mediated signalling is preferably effective to sensitize the cancer to (i.e. render the cancer susceptible to) treatment with a HER3-binding antigen-binding molecule, such that treatment of the subject's cancer with a HER3-binding antigen-binding molecule is more effective than it would have been in the absence of treatment with an antagonist of HER3-mediated signalling.

It will be appreciated that the particular antagonist of HER3-mediated signalling to be employed in a combination treatment according to the present disclosure may be selected in accordance with the mutation status/genotype of the cancer to be treated. By way of illustration, where a cancer comprises an activating mutation to KRAS, the antagonist may be an antagonist of signalling through the MAPK/ERK pathway. Similarly, where a cancer comprises an activating mutation to PIK3CA or an inactivating mutation to PTEN, the antagonist may be an antagonist of signalling through the PI3K/AKT/mTOR pathway.

In some embodiments, an antagonist of HER3-mediated signalling according to the present disclosure is a pan-ErbB inhibitor (e.g. sapitinib or Sym013). In some embodiments, an antagonist of HER3-mediated signalling is an inhibitor of signalling mediated by EGFR (e.g. cetuximab, panitumumab, gefitinib, erlotinib, lapatinib, afatinib, brigatinib, icotinib, osimertinib, zalutumumab, vandetanib, necitumumab, nimotuzumab, dacomitinib, duligotuzumab or matuzumab). In some embodiments, an antagonist of HER3-mediated signalling is an inhibitor of signalling mediated by HER2 (e.g. trastuzumab, pertuzumab, lapatinib, neratinib, afatinib, dacomitinib, MM-111, zenocutuzumab, MCLA-128 or margetuximab). In some embodiments, an antagonist of HER3-mediated signalling is an inhibitor of signalling mediated by HER3 (e.g. seribantumab, lumretuzumab, elgemtumab, KTN3379, AV-203, GSK2849330, REGN1400, MP-RM-1, EV20, pertuzumab, duligotuzumab, MM-111, zenocutuzumab, istiratumab, MCLA-128, patritumab, EZN-3920, RB200, U3-1402, TX2-121-2, EZN-3920 and miR-205). In some embodiments, an antagonist of HER3-mediated signalling is an inhibitor of signalling mediated by HER4 (e.g. lapatinib, ibrutinib, afatinib, dacomitinib or neratinib).

In some embodiments, an antagonist of HER3-mediated signalling inhibits a downstream effector of HER3 signalling. Downstream effectors of HER3-mediated signalling include e.g. PI3K, AKT, KRAS, BRAF, MEK/ERK and mTOR. In some embodiments, an antagonist of HER3-mediated signalling is an inhibitor of the MAPK/ERK pathway. In some embodiments, an antagonist of HER3-mediated signalling is an inhibitor of the PI3K/ATK/mTOR pathway.

In some embodiments, an antagonist of HER3-mediated signalling is a PI3K inhibitor (e.g. pictilisib, buparlisib, dactolisib, SAR245409, AZD8186, idelalisib, copanlisib or duvelisib). In some embodiments, an antagonist of HER3-mediated signalling is an AKT inhibitor (e.g. MK-2206, AZD5363, GSK690693, GSK2110183, ipatasertib, VQD-002, perifosine or miltefosine). In some embodiments, an antagonist of HER3-mediated signalling is an inhibitor of KRAS (e.g. sotorasib (also known as AMG 510), ARS-1620, adagrasib (also known as MRTX849), LY3499446, ARS-3248/JNJ-74699157, B12852 or RRSP chimeric toxin). In some embodiments, an antagonist of HER3-mediated signalling is a BRAF inhibitor (e.g. vemurafenib, dabrafenib, SB590885, XL281, RAF265, encorafenib, belvarafenib, PLX8394, LY3009120, LXH254, GDC-0879, PLX-4720, sorafenib, or LGX818). In some embodiments, an antagonist of HER3-mediated signalling is a MEK/ERK inhibitor (e.g. trametinib, cobimetinib, binimetinib, selumetinib, pimasertib, PD-325901, CI-1040, PD035901, or TAK-733). In some embodiments, an antagonist of HER3-mediated signalling is a mTOR inhibitor (e.g. rapamycin, deforolimus, temsirolimus, everolimus, ridaforolimus or sapanisertib).

### Therapeutic and Prophylactic Intervention

Aspects and embodiments of the present disclosure relate to therapeutic and prophylactic intervention for the treatment and prevention of the cancers described herein.

The present disclosure provides an antigen-binding molecule which binds to HER3 for use in a method of treating or preventing a cancer as described herein in a subject. Also provided is the use of an antigen-binding molecule which binds to HER3 in the manufacture of a medicament for use in treating or preventing a cancer as described herein in a subject. Also provided is a method of treating or preventing a cancer as described herein in a subject, comprising administering to the subject a therapeutically- or prophylactically-effective amount of an antigen-binding molecule which binds to HER3.

The present disclosure also provides an antigen-binding molecule which binds to HER3 for use in a method of treating or preventing a cancer as described herein in a subject, wherein the method further comprises administering an antagonist of HER3-mediated signalling. Also provided is the use of an antigen-binding molecule which binds to HER3 in the manufacture of a medicament for use in treating or preventing a cancer as described herein in a subject, wherein the method further comprises administering an antagonist of HER3-mediated signalling. Also provided is a method of treating or preventing a cancer as described herein in a subject, comprising administering to the subject a therapeutically- or prophylactically-effective amount of an antigen-binding molecule which binds to HER3, wherein the method further comprises administering an antagonist of HER3-mediated signalling.

The present disclosure also provides (i) an antagonist of HER3-mediated signalling and (ii) an antigen-binding molecule which binds to HER3 for use in a method of treating or preventing a cancer as described herein in a subject. Also provided is the use of (i) an antagonist of HER3-mediated signalling and (ii) an antigen-binding molecule which binds to HER3 in the manufacture of a medicament for use in treating or preventing a cancer as described herein in a subject. Also provided is a method of treating or preventing a cancer as described herein in a subject, comprising administering to the subject a therapeutically- or prophylactically-effective amount of (i) an antagonist of HER3-mediated signalling and (ii) an antigen-binding molecule which binds to HER3.

In embodiments in accordance with aspects of the preceding paragraph, provision of (i) and (ii) may be as a combination therapy. In some embodiments, (i) and (ii) may be provided simultaneously or sequentially.

The present disclosure also provides an antagonist of HER3-mediated signalling for use in a method of treating or preventing a cancer as described herein in a subject, wherein the method further comprises administering an antigen-binding molecule which binds to HER3. Also provided is the use of an antagonist of HER3-mediated signalling in the manufacture of a medicament for use in treating or preventing a cancer as described herein in a subject, wherein the method further comprises administering an antigen-binding molecule which binds to HER3. Also provided is a method of treating or preventing a cancer as described herein in a subject, comprising administering to the subject a therapeutically- or prophylactically-effective amount of an antagonist of HER3-mediated signalling wherein the method further comprises administering an antigen-binding molecule which binds to HER3.

Therapeutic or prophylactic intervention in accordance with the present disclosure may be effective to reduce the development or progression of the cancer, alleviate one or more symptoms of the cancer or reduce the pathology of cancer. The intervention may be effective to prevent progression of the cancer, e.g. to prevent worsening of, or to slow the rate of development of, the cancer. In some embodiments, the intervention may lead to an improvement in the cancer, e.g. a reduction in the symptoms of the cancer or a reduction in some other correlate of the severity/activity of the cancer. In some embodiments, the methods may prevent development of the cancer to a later stage (e.g. a more severe stage or metastasis).

In some embodiments, the therapeutic or prophylactic intervention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

Administration of the articles of the present disclosure (i.e. the HER3-binding antigen-binding molecules and antagonists of HER3-mediated signalling) is preferably in a “therapeutically-effective” or “prophylactically-effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be by any suitable route, e.g. injection or infusion (e.g. via cannula). Administration may be to the blood, a tumor, or to a selected region of the human or animal body (e.g. an organ/tissue in which symptoms of the cancer manifest). The particular mode and/or site of administration may be selected in accordance with the location where the treatment effect is required.

Where two or more agents (e.g. an antagonist of HER3-mediated signalling and a HER3-binding antigen-binding molecule) are administered in combination, the agents may be administered either simultaneously or sequentially.

Simultaneous administration refers to administration of the two or more agents (e.g. an antagonist of HER3-mediated signalling and a HER3-binding antigen-binding molecule) together, for example as a pharmaceutical composition containing both agents (combined preparation), or immediately after each other (e.g. within 1, 4, 6, 8 or 12 hours), and optionally via the same route of administration, e.g. to the same artery, vein or other blood vessel.

Sequential administration refers to administration of one of the agents followed after a given time interval by separate administration of another agent. It is not required that the agents are administered by the same route, although this is the case in some embodiments. The time interval may be any time interval.

In some embodiments, therapeutic or prophylactic intervention according to the present disclosure comprises: (i) administering an antagonist of HER3-mediated signalling (e.g. an antagonist of HER3-mediated signalling described herein) to a subject having a cancer (e.g. a cancer comprising/heterozygous for/homozygous for a mutation resulting in upregulation of HER3-mediated signalling as described herein), and (ii) administering to the subject an antigen-binding molecule which binds to HER3 (e.g. a HER3-binding antigen-binding molecule as described herein). In some embodiments, (i) and (ii) are performed simultaneously. In some embodiments, (i) and (ii) are performed sequentially (e.g. (i) may be followed by (ii), or (ii) may be followed by (i)).

In some embodiments, the intervention comprises additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments, the therapeutic or prophylactic intervention comprises a stem cell transplant.

Multiple doses of the articles of the present disclosure may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days). One or more, or each, of the doses may be accompanied by simultaneous or sequential administration of another therapeutic agent.

### Diagnostic and Prognostic Methods, and Patient Selection

The present disclosure also provides diagnostic, prognostic and predictive methods in connection with the cancers described herein.

The methods may be performed in vitro on a sample obtained from a subject, or following processing of a sample obtained from a subject. Once the sample is collected, the subject is not required to be present for the in vitro method to be performed, and therefore the method may be one which is not practised on the human or animal body. However, in some embodiments the methods may be performed in vivo.

A sample may be taken from any tissue or bodily fluid. A sample may comprise or may be derived from: a quantity of blood; a quantity of serum derived from a subject's blood which may comprise the fluid portion of the blood obtained after removal of the fibrin clot and blood cells; a tissue sample or biopsy; pleural fluid; cerebrospinal fluid (CSF); or cells isolated from a subject. In some embodiments, the sample may be obtained or derived from a tissue or tissues which are affected by the disease/condition (e.g. tissue or tissues in which symptoms of the disease manifest, or which are involved in the pathogenesis of the disease/condition). In some embodiments, the sample may be obtained or derived from a cancer, tumor, or cells thereof.

The methods may be performed for the purpose of diagnosing a cancer (e.g. a cancer described herein). The methods may be performed for the purpose of prognosing/predicting the likely response of a subject to therapeutic/prophylactic intervention as described herein. The methods may be useful to predict the likely response to a given therapeutic/prophylactic intervention, e.g. in terms of efficacy, and may therefore by useful for supporting clinical decision-making. The methods may be performed for the purpose of identifying/selecting a subject for therapeutic/prophylactic intervention as described herein.

In some aspects and embodiments, the methods comprise analysing a subject's cancer in order to determine whether the cancer is a cancer described herein, e.g. a cancer not homozygous for/heterozygous for/comprising a mutation resulting in upregulation of HER3-mediated signalling, or a cancer comprising/heterozygous for/homozygous for a mutation resulting in upregulation of HER3-mediated signalling.

In some embodiments, the methods comprise evaluating a cancer in order to determine whether it comprises one or more mutations as described hereinabove. In some embodiments, the methods comprise evaluating a cancer in order to determine whether it comprises cells comprising one or more copies of an allele comprising a mutation as described hereinabove.

A cancer which is identified following such analysis not to comprise the mutation(s), and/or not to comprise cells comprising one or more copies of allele(s) comprising the mutation(s), may be identified as a cancer suitable for intervention with an antigen-binding molecule which binds to HER3 according to the present disclosure.

A cancer which is identified following such analysis to comprise the mutation(s), and/or to comprise cells comprising one or more copies of allele(s) comprising the mutation(s), may be identified as a cancer suitable for intervention with the combination of (i) an antagonist of HER3-mediated signalling according to the present disclosure, and (ii) an antigen-binding molecule which binds to HER3 according to the present disclosure.

Aspects and embodiments of the present disclosure also comprise selecting a subject for therapeutic or prophylactic intervention in accordance with the present disclosure.

A subject having a cancer which is identified following such analysis not to comprise the mutation(s), and/or not to comprise cells comprising one or more copies of allele(s) comprising the mutation(s), may be selected for treatment using an antigen-binding molecule which binds to HER3 according to the present disclosure.

A subject having a cancer which is identified following such analysis to comprise the mutation(s), and/or to comprise cells comprising one or more copies of allele(s) comprising the mutation(s) may be selected for treatment using (i) an antagonist of HER3-mediated signalling according to the present disclosure, and (ii) an antigen-binding molecule which binds to HER3 according to the present disclosure.

In some embodiments, a subject is selected/not selected for therapeutic intervention in accordance with the present disclosure based on the result of analysis of their cancer to determine whether the cancer comprises a mutation described herein, and/or cells comprising one or more copies of an allele comprising a mutation described herein.

In some embodiments, a method comprises selecting a subject for treatment with an antigen-binding molecule which binds to HER3 according to the present disclosure based on determination that the subject has a cancer which does not comprise a mutation described herein, and/or that cells of the subject's cancer are not determined to comprise one or more copies of an allele comprising a mutation described herein.

In some embodiments, a method comprises selecting a subject for treatment with (i) an antagonist of HER3-mediated signalling according to the present disclosure, and (ii) an antigen-binding molecule which binds to HER3 according to the present disclosure, based on determination that the subject has a cancer comprising a mutation described herein, and/or that cells of the cancer comprise one or more copies of an allele comprising a mutation described herein.

In some aspects and embodiments, the methods comprise:
(a) analysing a subject's cancer in order to determine whether the cancer comprises a mutation described herein, and/or one or more copies of an allele comprising a mutation described herein; and
(b) selecting a subject for treatment with an antigen-binding molecule which binds to HER3 according to the present disclosure where the subject's cancer is determined in step (a) not to be homozygous for/heterozygous for or not to comprise a mutation described herein (e.g. where the subject's cancer is determined to be a cancer according to an embodiment of a cancer described in the section entitled “cancers not homozygous for/heterozygous for/comprising mutations resulting in upregulation of HER3-mediated signalling”).

In some embodiments, the methods further comprise:
(c) administering an antigen-binding molecule which binds to HER3 according to the present disclosure to a subject selected for treatment in step (b).

In some aspects and embodiments, the methods comprise:
(a) analysing a subject's cancer in order to determine whether the cancer comprises a mutation described herein, and/or one or more copies of an allele comprising a mutation described herein; and
(b) selecting a subject for treatment with (i) an antagonist of HER3-mediated signalling according to the present disclosure, and (ii) an antigen-binding molecule which binds to HER3 according to the present disclosure, where the subject's cancer is determined in step (a) to comprise or to be heterozygous for/homozygous for a mutation described herein (e.g. where the subject's cancer is determined to be a cancer according to an embodiment of a cancer described in the section entitled “cancers comprising/heterozygous for/homozygous for mutations resulting in upregulation of HER3-mediated signalling”).

In some embodiments, the methods further comprise:
(c) administering (i) an antagonist of HER3-mediated signalling according to the present disclosure, and (ii) an antigen-binding molecule which binds to HER3 according to the present disclosure, to a subject selected for treatment in step (b).

In some aspects and embodiments, the methods comprise:
(a) analysing a subject's cancer in order to determine whether the cancer comprises a mutation described herein, and/or one or more copies of an allele comprising a mutation described herein;
(b) selecting a subject for treatment with (i) an antagonist of HER3-mediated signalling according to the present disclosure, and (ii) an antigen-binding molecule which binds to HER3 according to the present disclosure, where the subject's cancer is determined in step (a) to comprise or to be heterozygous for/homozygous for a mutation described herein (e.g. where the subject's cancer is determined to be a cancer according to an embodiment of a cancer described in the section entitled “cancers comprising/heterozygous for/homozygous for mutations resulting in upregulation of HER3-mediated signalling”);
(c) administering an antagonist of HER3-mediated signalling according to the present disclosure to a subject selected for treatment in step (b); and
(d) administering an antigen-binding molecule which binds to HER3 according to the present disclosure to a subject selected for treatment in step (b).

It will be appreciated that steps (c) and (d) of the preceding paragraph may be performed simultaneously or sequentially.

Analysis of a cancer may comprise analysing the nucleotide sequence(s) of gene(s) encoding one or more factors involved in HER3-mediated signalling (e.g. in a nucleic acid-containing sample obtained from the cancer), in order to determine whether the cancer comprises mutation(s) as described hereinabove (e.g. mutations resulting in upregulation of HER3-mediated signalling, mutations resulting in upregulation of signalling through the MAPK/ERK pathway and/or mutations resulting in upregulation of signalling through the PI3K/AKT/mTOR pathway; e.g. activating mutations to genes encoding positive regulators of said signalling/signalling pathways, and/or inactivating mutations to genes encoding negative regulators of said signalling/signalling pathways). Analysis may be of cells of a cancer. Analysis may be of a nucleic acid-containing sample/biopsy obtained from a cancer/cells thereof. Such analysis is preferably performed in vitro.

Methods for evaluating the nucleotide sequence(s) of genes of interest are well known in the art, and include e.g. sequencing by the classic chain termination method, and next generation sequencing (NGS) technologies, which are reviewed e.g. by Metzker, M. L., Nat Rev Genet (2010) 11(1): 31-46 (hereby incorporated by reference in its entirety). Further methods for evaluating the nucleotide sequence(s) of genes of interest for the presence/absence of mutations include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, allele specific oligonucleotide (ASO) probes, and oligonucleotide microarrays or beads. Other suitable methods are described e.g. in Edenberg H J and Liu Y, Cold Spring Harb Protoc; 2009; doi:10.1101/pdb.top62, and Tsuchihashi Z and Dracopoli N C, Pharmacogenomics J., 2002, 2:103-110.

In further aspects, the present disclosure provides methods for determining whether a subject is likely to respond well to therapeutic or prophylactic intervention using an antigen-binding molecule which binds to HER3.

The response of a subject/cancer to a given therapeutic or prophylactic intervention may be evaluated in accordance with the Revised Criteria for Response Assessment: The Lugano Classification (described e.g. in Cheson et al., J Clin Oncol (2014) 32: 3059-3068, hereby incorporated by reference in its entirety). In some embodiments, a subject/cancer is considered to “respond” to a given intervention if one of the following is achieved: complete response, partial response, or stable disease. In some embodiments, a subject/cancer is considered to “respond” to a given intervention if one of the following is achieved: complete response or partial response.

In some embodiments, the methods comprise: analysing a subject's cancer in order to determine whether the cancer comprises a mutation described herein, and/or one or more copies of an allele comprising a mutation described herein; wherein a subject having a cancer determined to comprise or to be heterozygous for/homozygous for a mutation described herein (e.g. where the subject's cancer is determined to be a cancer according to an embodiment of a cancer described in the section entitled “cancers comprising/heterozygous for/homozygous for mutations resulting in upregulation of HER3-mediated signalling”) is determined not to be likely to respond well to therapeutic or prophylactic intervention using an antigen-binding molecule which binds to HER3.

In some embodiments, the methods comprise: analysing a subject's cancer in order to determine whether the cancer comprises a mutation described herein, and/or one or more copies of an allele comprising a mutation described herein; wherein a subject having a cancer determined not to be homozygous for/heterozygous for or not to comprise a mutation described herein (e.g. where the subject's cancer is determined to be a cancer according to an embodiment of a cancer described in the section entitled “cancers not homozygous for/heterozygous for/comprising mutations resulting in upregulation of HER3-mediated signalling”), is determined to be likely to respond well to therapeutic or prophylactic intervention using an antigen-binding molecule which binds to HER3.

### Subjects

The subject in accordance with aspects the present disclosure may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer described herein), may be suspected of having such a disease/condition, or may be at risk of developing/contracting such a disease/condition.

The subject may have a cancer. The subject may have (e.g. may have been determined to have) a cancer which is not homozygous for, which is not heterozygous for, or which does not comprise one or more mutations resulting in upregulation of HER3-mediated signalling, as described herein. The subject may have (e.g. may have been determined to have) a cancer which comprises, is heterozygous for, or which is homozygous for one or more mutations resulting in upregulation of HER3-mediated signalling, as described herein.

### Kits

The present disclosure also provides kits of parts. A kit according to the present disclosure may comprise components for performing a method described herein, in whole or in part.

In some embodiments, a kit may comprise: (i) means for evaluating a cancer in order to determine whether it comprises one or more mutations as described herein, and (ii) an antigen-binding molecule which binds to HER3 as described herein. In some embodiments, a kit may comprise an antagonist of HER3-mediated signalling as described herein.

Means for evaluating a cancer in order to determine whether it comprises one or more mutations described herein may comprise e.g. one or more oligonucleotides having complementarity to the nucleotide sequence of gene(s) described herein.

The kit may comprise instructions for evaluation of a cancer in order to determine whether it comprises one or more mutations as described herein. The kit may comprise instructions for administering an antigen-binding molecule which binds to HER3 as described herein to a subject. The kit may further comprise instructions for administering an antagonist of HER3-mediated signalling as described herein to a subject.

The kit may further comprise reagents, buffers and/or standards required for execution of a method according to the present disclosure.

### Sequence Identity

As used herein, “sequence identity” refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al., 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

Sequences

| SEQ | | |
|---|---|---|
| ID NO: | DESCRIPTION | SEQUENCE |
| 1 | Human HER3 | MRANDALQVLGLLFSLARGSEVGNSQAVCPGTLNGLSVTGDAENQYQTLYKLYERCEVVMGNLEIVLTGHN |
| | isoform 1 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-1, v1) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKACEGTGSGSRFQTVDSSNIDGFVN |
| | | CTKILGNLDFLITGLNGDPWHKIPALDPEKLNVFRTVREITGYLNIQSWPPHMHNFSVFSNLTTIGGRSLYNRG |
| | | FSLLIMKNLNVTSLGFRSLKEISAGRIYISANRQLCYHHSLNWTKVLRGPTEERLDIKHNRPRRDCVAEGKVC |
| | | DPLCSSGGCWGPGPGQCLSCRNYSRGGVCVTHCNFLNGEPREFAHEAECFSCHPECQPMEGTATCNGS |
| | | GSDTCAQCAHFRDGPHCVSSCPHGVLGAKGPIYKYPDVQNECRPCHENCTQGCKGPELQDCLGQTLVLIG |
| | | KTHLTMALTVIAGLVVIFMMLGGTFLYWRGRRIQNKRAMRRYLERGESIEPLDPSEKANKVLARIFKETELRK |
| | | LKVLGSGVFGTVHKGVWIPEGESIKIPVCIKVIEDKSGRQSFQAVTDHMLAIGSLDHAHIVRLLGLCPGSSLQL |
| | | VTQYLPLGSLLDHVRQHRGALGPQLLLNWGVQIAKGMYYLEEHGMVHRNLAARNVLLKSPSQVQVADFGV |
| | | ADLLPPDDKQLLYSEAKTPIKWMALESIHFGKYTHQSDVWSYGVTVWELMTFGAEPYAGLRLAEVPDLLEK |
| | | GERLAQPQICTIDVYMVMVKCWMIDENIRPTFKELANEFTRMARDPPRYLVIKRESGPGIAPGPEPHGLTNK |
| | | KLEEVELEPELDLDLDLEAEEDNLATTTLGSALSLPVGTLNRPRGSQSLLSPSSGYMPMNQGNLGESCQES |
| | | AVSGSSERCPRPVSLHPMPRGCLASESSEGHVTGSEAELQEKVSMCRSRSRSRSPRPRGDSAYHSQRHS |
| | | LLTPVTPLSPPGLEEEDVNGYVMPDTHLKGTPSSREGTLSSVGLSSVLGTEEEDEDEEYEYMNRRRRHSPP |
| | | HPPRPSSLEELGYEYMDVGSDLSASLGSTQSCPLHPVPIMPTAGTTPDEDYEYMNRQRDGGGPGGDYAA |
| | | MGACPASEQGYEEMRAFQGPGHQAPHVHYARLKTLRSLEATDSAFDNPDYWHSRLFPKANAQRT |
| 2 | Human HER3 | MRANDALQVLGLLFSLARGSEVGNSQAVCPGTLNGLSVTGDAENQYQTLYKLYERCEVVMGNLEIVLTGHN |
| | isoform 2 | ADLSFLQWIREVTGYVLVAMNEFSTLPLPNLRVVRGTQVYDGKFAIFVMLNYNTNSSHALRQLRLTQLTGQF |
| | (UniProt: | PMVPSGLTPQPAQDWYLLDDDPRLLTLSASSKVPVTLAAV |
| | P21860-2) | |
| 3 | Human HER3 | MRANDALQVLGLLFSLARGSEVGNSQAVCPGTLNGLSVTGDAENQYQTLYKLYERCEVVMGNLEIVLTGHN |
| | isoform 3 | ADLSFLQWIREVTGYVLVAMNEFSTLPLPNLRVVRGTQVYDGKFAIFVMLNYNTNSSHALRQLRLTQLTEILS |
| | (UniProt: | GGVYIEKNDKLCHMDTIDWRDIVRDRDAEIVVKDNGRSCPPCHEVCKGRCWGPGSEDCQTLTKTICAPQCN |
| | P21860-3) | GHCFGPNPNQCCHDECAGGCSGPQDTDCFACRHFNDSGACVPRCPQPLVYNKLTFQLEPNPHTKYQYGG |
| | | VCVASCPHNFVVDQTSCVRACPPDKMEVDKNGLKMCEPCGGLCPKAF |
| 4 | Human HER3 | MGNLEIVLTGHNADLSFLQWIREVTGYVLVAMNEFSTLPLPNLRVVRGTQVYDGKFAIFVMLNYNTNSSHAL |
| | isoform 4 | RQLRLTQLTEILSGGVYIEKNDKLCHMDTIDWRDIVRDRDAEIVVKDNGRSCPPCHEVCKGRCWGPGSEDC |
| | (UniProt: | QTLTKTICAPQCNGHCFGPNPNQCCHDECAGGCSGPQDTDCFACRHFNDSGACVPRCPQPLVYNKLTFQL |
| | P21860-4) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMHNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKVIEDKSGRQSFQAVTDHMLAIGSLDHAHIVRL |
| | | LGLCPGSSLQLVTQYLPLGSLLDHVRQHRGALGPQLLLNWGVQIAKGMYYLEEHGMVHRNLAARNVLLKSP |
| | | SQVQVADFGVADLLPPDDKQLLYSEAKTPIKWMALESIHFGKYTHQSDVWSYGVTVWELMTFGAEPYAGLR |
| | | LAEVPDLLEKGERLAQPQICTIDVYMVMVKCWMIDENIRPTFKELANEFTRMARDPPRYLVIKRESGPGIAPG |
| | | PEPHGLTNKKLEEVELEPELDLDLDLEAEEDNLATTTLGSALSLPVGTLNRPRGSQSLLSPSSGYMPMNQGN |
| | | LGESCQESAVSGSSERCPRPVSLHPMPRGCLASESSEGHVTGSEAELQEKVSMCRSRSRSRSPRPRGDS |
| | | AYHSQRHSLLTPVTPLSPPGLEEEDVNGYVMPDTHLKGTPSSREGTLSSVGLSSVLGTEEEDEDEEYEYMN |
| | | RRRRHSPPHPPRPSSLEELGYEYMDVGSDLSASLGSTQSCPLHPVPIMPTAGTTPDEDYEYMNRQRDGGG |
| | | PGGDYAAMGACPASEQGYEEMRAFQGPGHQAPHVHYARLKTLRSLEATDSAFDNPDYWHSRLFPKANAQ |
| | | RT |
| 5 | Human HER3 | MALTVIAGLVVIFMMLGGTFLYWRGRRIQNKRAMRRYLERGESIEPLDPSEKANKVLARIFKETELRKLKVLG |
| | isoform 5 | SGVFGTVHKGVWIPEGESIKIPVCIKVIEDKSGRQSFQAVTDHMLAIGSLDHAHIVRLLGLCPGSSLQLVTQYL |
| | (UniProt: | PLGSLLDHVRQHRGALGPQLLLNWGVQIAKGMYYLEEHGMVHRNLAARNVLLKSPSQVQVADFGVADLLP |
| | P21860-5) | PDDKQLLYSEAKTPIKWMALESIHFGKYTHQSDVWSYGVTVWELMTFGAEPYAGLRLAEVPDLLEKGERLA |
| | | QPQICTIDVYMVMVKCWMIDENIRPTFKELANEFTRMARDPPRYLVIKRESGPGIAPGPEPHGLTNKKLEEVE |
| | | LEPELDLDLDLEAEEDNLATTTLGSALSLPVGTLNRPRGSQSLLSPSSGYMPMNQGNLGESCQESAVSGSS |
| | | ERCPRPVSLHPMPRGCLASESSEGHVTGSEAELQEKVSMCRSRSRSRSPRPRGDSAYHSQRHSLLTPVTP |
| | | LSPPGLEEEDVNGYVMPDTHLKGTPSSREGTLSSVGLSSVLGTEEEDEDEEYEYMNRRRRHSPPHPPRPS |
| | | SLEELGYEYMDVGSDLSASLGSTQSCPLHPVPIMPTAGTTPDEDYEYMNRQRDGGGPGGDYAAMGACPA |
| | | SEQGYEEMRAFQGPGHQAPHVHYARLKTLRSLEATDSAFDNPDYWHSRLFPKANAQRT |
| 6 | Mature human | SEVGNSQAVCPGTLNGLSVTGDAENQYQTLYKLYERCEVVMGNLEIVLTGHNADLSFLQWIREVTGYVLVA |
| | HER3 isoform 1 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-1, v1 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKACEGTGSGSRFQTVDSSNIDGFVNCTKILGNLDFLITGLNGDP |
| | 1342) | WHKIPALDPEKLNVFRTVREITGYLNIQSWPPHMHNFSVFSNLTTIGGRSLYNRGFSLLIMKNLNVTSLGFRS |
| | | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLTMALTVIAGLVVIFM |
| | | MLGGTFLYWRGRRIQNKRAMRRYLERGESIEPLDPSEKANKVLARIFKETELRKLKVLGSGVFGTVHKGVWI |
| | | PEGESIKIPVCIKVIEDKSGRQSFQAVTDHMLAIGSLDHAHIVRLLGLCPGSSLQLVTQYLPLGSLLDHVRQHR |
| | | GALGPQLLLNWGVQIAKGMYYLEEHGMVHRNLAARNVLLKSPSQVQVADFGVADLLPPDDKQLLYSEAKTP |
| | | IKWMALESIHFGKYTHQSDVWSYGVTVWELMTFGAEPYAGLRLAEVPDLLEKGERLAQPQICTIDVYMVMV |
| | | KCWMIDENIRPTFKELANEFTRMARDPPRYLVIKRESGPGIAPGPEPHGLTNKKLEEVELEPELDLDLDLEAE |
| | | EDNLATTTLGSALSLPVGTLNRPRGSQSLLSPSSGYMPMNQGNLGESCQESAVSGSSERCPRPVSLHPMP |
| | | RGCLASESSEGHVTGSEAELQEKVSMCRSRSRSRSPRPRGDSAYHSQRHSLLTPVTPLSPPGLEEEDVNG |
| | | YVMPDTHLKGTPSSREGTLSSVGLSSVLGTEEEDEDEEYEYMNRRRRHSPPHPPRPSSLEELGYEYMDVG |
| | | SDLSASLGSTQSCPLHPVPIMPTAGTTPDEDYEYMNRQRDGGGPGGDYAAMGACPASEQGYEEMRAFQG |
| | | PGHQAPHVHYARLKTLRSLEATDSAFDNPDYWHSRLFPKANAQRT |
| 7 | Mature human | SEVGNSQAVCPGTLNGLSVTGDAENQYQTLYKLYERCEVVMGNLEIVLTGHNADLSFLQWIREVTGYVLVA |
| | HER3 isoform 2 | MNEFSTLPLPNLRVVRGTQVYDGKFAIFVMLNYNTNSSHALRQLRLTQLTGQFPMVPSGLTPQPAQDWYLL |
| | (UniProt: | DDDPRLLTLSASSKVPVTLAAV |
| | P21860-2 | |
| | positions 20 to | |
| | 183) | |
| 8 | Mature human | SEVGNSQAVCPGTLNGLSVTGDAENQYQTLYKLYERCEVVMGNLEIVLTGHNADLSFLQWIREVTGYVLVA |
| | HER3 isoform 3 | MNEFSTLPLPNLRVVRGTQVYDGKFAIFVMLNYNTNSSHALRQLRLTQLTEILSGGVYIEKNDKLCHMDTIDW |
| | (UniProt: | RDIVRDRDAEIVVKDNGRSCPPCHEVCKGRCWGPGSEDCQTLTKTICAPQCNGHCFGPNPNQCCHDECA |
| | P21860-3 | GGCSGPQDTDCFACRHFNDSGACVPRCPQPLVYNKLTFQLEPNPHTKYQYGGVCVASCPHNFVVDQTSC |
| | positions 20 to | VRACPPDKMEVDKNGLKMCEPCGGLCPKAF |
| | 331) | |
| 9 | Human HER3 | SEVGNSQAVCPGTLNGLSVTGDAENQYQTLYKLYERCEVVMGNLEIVLTGHNADLSFLQWIREVTGYVLVA |
| | isoform 1 | MNEFSTLPLPNLRVVRGTQVYDGKFAIFVMLNYNTNSSHALRQLRLTQLTEILSGGVYIEKNDKLCHMDTIDW |
| | extracellular | RDIVRDRDAEIVVKDNGRSCPPCHEVCKGRCWGPGSEDCQTLTKTICAPQCNGHCFGPNPNQCCHDECA |
| | region (UniProt: | GGCSGPQDTDCFACRHFNDSGACVPRCPQPLVYNKLTFQLEPNPHTKYQYGGVCVASCPHNFVVDQTSC |
| | P21860-1, v1 | VRACPPDKMEVDKNGLKMCEPCGGLCPKACEGTGSGSRFQTVDSSNIDGFVNCTKILGNLDFLITGLNGDP |
| | positions 20 to | WHKIPALDPEKLNVFRTVREITGYLNIQSWPPHMHNFSVFSNLTTIGGRSLYNRGFSLLIMKNLNVTSLGFRS |
| | 643) | LKEISAGRIYISANRQLCYHHSLNWTKVLRGPTEERLDIKHNRPRRDCVAEGKVCDPLCSSGGCWGPGPGQ |
| | | CLSCRNYSRGGVCVTHCNFLNGEPREFAHEAECFSCHPECQPMEGTATCNGSGSDTCAQCAHFRDGPHC |
| | | VSSCPHGVLGAKGPIYKYPDVQNECRPCHENCTQGCKGPELQDCLGQTLVLIGKTHLT |
| 10 | Human HER3 | MALTVIAGLVVIFMMLGGTFL |
| | isoform 1 | |
| | transmembrane | |
| | domain (UniProt: | |
| | P21860-1, v1 | |
| | positions 644 to | |
| | 664) | |
| 11 | Human HER3 | YWRGRRIQNKRAMRRYLERGESIEPLDPSEKANKVLARIFKETELRKLKVLGSGVFGTVHKGVWIPEGESIKI |
| | isoform 1 | PVCIKVIEDKSGRQSFQAVTDHMLAIGSLDHAHIVRLLGLCPGSSLQLVTQYLPLGSLLDHVRQHRGALGPQL |
| | cytoplasmic | LLNWGVQIAKGMYYLEEHGMVHRNLAARNVLLKSPSQVQVADFGVADLLPPDDKQLLYSEAKTPIKWMALE |
| | domain (UniProt: | SIHFGKYTHQSDVWSYGVTVWELMTFGAEPYAGLRLAEVPDLLEKGERLAQPQICTIDVYMVMVKCWMIDE |
| | P21860-1, v1 | NIRPTFKELANEFTRMARDPPRYLVIKRESGPGIAPGPEPHGLTNKKLEEVELEPELDLDLDLEAEEDNLATTT |
| | positions 665 to | LGSALSLPVGTLNRPRGSQSLLSPSSGYMPMNQGNLGESCQESAVSGSSERCPRPVSLHPMPRGCLASES |
| | 1342) | SEGHVTGSEAELQEKVSMCRSRSRSRSPRPRGDSAYHSQRHSLLTPVTPLSPPGLEEEDVNGYVMPDTHL |
| | | KGTPSSREGTLSSVGLSSVLGTEEEDEDEEYEYMNRRRRHSPPHPPRPSSLEELGYEYMDVGSDLSASLG |
| | | STQSCPLHPVPIMPTAGTTPDEDYEYMNRQRDGGGPGGDYAAMGACPASEQGYEEMRAFQGPGHQAPH |
| | | VHYARLKTLRSLEATDSAFDNPDYWHSRLFPKANAQRT |
| 12 | Human HER3 | YWRGRRIQNKRAMRRYLERGESIEPLDPSEKANKVLARIFKETE |
| | isoform 1 | |
| | juxtamembrane | |
| | segment | |
| | (UniProt: | |
| | P21860-1, V1 | |
| | positions 665 to | |
| | 708) | |
| 13 | Human HER3 | LRKLKVLGSGVFGTVHKGVWIPEGESIKIPVCIKVIEDKSGRQSFQAVTDHMLAIGSLDH |
| | isoform 1 protein | AHIVRLLGLCPGSSLQLVTQYLPLGSLLDHVRQHRGALGPQLLLNWGVQIAKGMYYLEEH |
| | kinase domain | GMVHRNLAARNVLLKSPSQVQVADFGVADLLPPDDKQLLYSEAKTPIKWMALESIHFGKY |
| | (UniProt: | THQSDVWSYGVTVWELMTFGAEPYAGLRLAEVPDLLEKGERLAQPQICTIDVYMVMVKCW |
| | P21860-1, v1 | MIDENIRPTFKELANEFT |
| | positions 709 to | |
| | 966) | |
| 14 | Human HER3 | RMARDPPRYLVIKRESGPGIAPGPEPHGLTNKKLEEVELEPELDLDLDLEAEEDNLATTTLGSALSLPVGTLN |
| | isoform 1 C | RPRGSQSLLSPSSGYMPMNQGNLGESCQESAVSGSSERCPRPVSLHPMPRGCLASESSEGHVTGSEAEL |
| | terminal | QEKVSMCRSRSRSRSPRPRGDSAYHSQRHSLLTPVTPLSPPGLEEEDVNGYVMPDTHLKGTPSSREGTLS |
| | segment | SVGLSSVLGTEEEDEDEEYEYMNRRRRHSPPHPPRPSSLEELGYEYMDVGSDLSASLGSTQSCPLHPVPIM |
| | (UniProt: | PTAGTTPDEDYEYMNRQRDGGGPGGDYAAMGACPASEQGYEEMRAFQGPGHQAPHVHYARLKTLRSLE |
| | P21860-1, v1 | ATDSAFDNPDYWHSRLFPKANAQRT |
| | positions 967 to | |
| | 1342) | |
| 15 | Human HER3 | SEVGNSQAVCPGTLNGLSVTGDAENQYQTLYKLYERCEVVMGNLEIVLTGHNADLSFLQWIREVTGYVLVA |
| | extracellular | MNEFSTLPLPNLRVVRGTQVYDGKFAIFVMLNYNTNSSHALRQLRLTQLTEILSGGVYIEKNDKLCHMDTIDW |
| | region | RDIVRDRDAEIVVKDNGRSC |
| | subdomain I | |
| | (UniProt: | |
| | P21860-1, v1 | |
| | positions 20 to | |
| | 183) | |
| 16 | Human HER3 | PPCHEVCKGRCWGPGSEDCQTLTKTICAPQCNGHCFGPNPNQCCHDECAGGCSGPQDTDCFACRHFND |
| | extracellular | SGACVPRCPQPLVYNKLTFQLEPNPHTKYQYGGVCVASCPHNFVVDQTSCVRACPPDKMEVDKNGLKMC |
| | region | EPCGGLCPK |
| | subdomain II | |
| | (UniProt: | |
| | P21860-1, v1 | |
| | positions 184 to | |
| | 329 | |
| 17 | Human HER3 | ACEGTGSGSRFQTVDSSNIDGFVNCTKILGNLDFLITGLNGDPWHKIPALDPEKLNVFRTVREITGYLNIQSW |
| | extracellular | PPHMHNFSVFSNLTTIGGRSLYNRGFSLLIMKNLNVTSLGFRSLKEISAGRIYISANRQLCYHHSLNWTKVLR |
| | region | GPTEERLDIKHNRPRRDCVA |
| | subdomain III | |
| | (UniProt: | |
| | P21860-1, v1 | |
| | positions 330 to | |
| | 495) | |
| 18 | Human HER3 | EGKVCDPLCSSGGCWGPGPGQCLSCRNYSRGGVCVTHCNFLNGEPREFAHEAECFSCHPECQPMEGTA |
| | extracellular | TCNGSGSDTCAQCAHFRDGPHCVSSCPHGVLGAKGPIYKYPDVQNECRPCHENCTQGCKGPELQDCLGQ |
| | region | TLVLIGKTHLT |
| | subdomain IV | |
| | (UniProt: | |
| | P21860-1, v1 | |
| | positions 496 to | |
| | 643) | |
| 19 | Human HER3 | QPLVYNKLTFQLEPNPH |
| | extracellular | |
| | region | |
| | subdomain II | |
| | dimerisation loop | |
| | (UniProt: | |
| | P21860-1, v1 | |
| | positions 261 to | |
| | 278) | |
| 20 | Rhesus | MGNLEIVLTGHNADLSFLQWIREVTGYVLVAMNEFSTLPLPNLRVVRGTQVYDGKFAIFVMLNYNTNSSHAL |
| | macaque HER3 | RQLRLTQLTEILSGGVYIEKNDKLCHMDTIDWKDIVRDQDAEIVVKDNGRSCPLCHEVCKGRCWGPGPEDC |
| | (UniProt: | QTLTKTICAPQCNGHCFGPNPNQCCHDECAGGCSGPQDTDCFACRHFNDSGACVPRCPQPLVYNKLTFQL |
| | F7HEH3-1, v2) | EPNPHTKYQYGGVCVASCPHNFVVDQTSCVRACPPDKMEVDKNGLKMCEPCGGLCPKACEGTGSGSRFQ |
| | | TVDSSNIDGFVNCTKILGNLDFLITGLNGDPWHKIPALDPEKLNVFRTVREITGYLNIQSWPPHMYNFSVFSNL |
| | | TTIGGRSLYNRGFSLLIMKNLNVTSLGFRSLKEISAGRIYISANRQLCYHHSLNWTKVLRGPTEERLDIKHNRP |
| | | RRDCVAEGKVCDPLCSSGGCWGPGPGQCLSCRNYSRGGVCVTHCNFLNGEPREFAHEAECFSCHPECQ |
| | | PMEGTATCNGSGSDTCAQCAHFRDGPHCVSSCPHGVLGAKGPIYKYPDVQNECRPCHENCTQGCKGPEL |
| | | QDCLGQTLVLIGKTHLTMALTVIAGLVVIFMMLGGTFLYWRGRRIQNKRAMRRYLERGESIEPLDPSEKANKV |
| | | LARIFKETELRKLKVLGSGVFGTVHKGVWIPEGESIKIPVCIKIIEDKSGRQSFQAVTDHMLAIGSLDHAHIVRLL |
| | | GLCPGSSLQLVTQYLPLGSLLDHVRQHRGALGPQLLLNWGVQIAKGMYYLEEHGMVHRNLAARNVLLKSPS |
| | | QVQVADFGVADLLPPDDKQLLYSEAKTPIKWMALESIHFGKYTHQSDVWSYGVTVWELMTFGAEPYAGLRL |
| | | AEVPDLLEKGERLAQPQICTIDVYMVMVKCWMIDENIRPTFKELANEFTRMARDPPRYLVIKRESGPGIAPGP |
| | | EPHGLTNKKLEEVELEPELDLDLDLEAEEDNLATTTLGSALSLPVGTLNRPRGSQSLLSPSSGYMPMNQGNL |
| | | GEAFQESAVSGSSEWCPRPVSLHPMPRGCLASESSEGHVTGSEAELQEKVSTCRSRSRSRSPRPRGDSA |
| | | YHSQRHSLLTPVTPLSPPGLEEEDVNGYVMPDTHLKGTPSSREGTLSSVGLSSVLGTEEEDEDEEYEYMNR |
| | | RRRHSPPRPPRPSSLEELGYEYMDVGSDLSASLGSTQSCPLHPVPVMPTAGTTPDEDYEYMNRQRGGSG |
| | | PGGDYAAMGACPASEQGYEEMRAFQGPGHQAPHVHYAHLKTLRSLEATDSAFDNPDYWHSRLFPKANAQ |
| | | RT |
| 21 | 10D1 heavy | DVQLQESGPDLVKPSQSLSLTCTVTGYSITSGYSWHWIRQFPGNKLEWMGSIHYSGGTNYNPSLKSRISITR |
| | chain variable | DTSKNQFFLQLNSVTTEDTATYFCARMTTAPRYPFDYWGQGTTLTVSS |
| | region | |
| 22 | 10D1_c75 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 23 | 10D1_c76 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 24 | 10D1_c77 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLKLSSVTAADTAVYFCARMTTAPRYPFDYWGQGTLVTVSS |
| | region | |
| 25 | 10D1_c78v1 | DVQLQEWGAGLLKPSETLSLTCAVTGYSITSGYSWHWIRQFPGNGLEWIGSIHYSGGTNYNPSLKSRITISR |
| | heavy chain | DTSKNQFSLKLSSVTAADTAVYFCARMTTAPRYPFDYWGQGTLVTVSS |
| | variable region | |
| 26 | 10D1_c78v2 | DVQLQEWGAGLLKPSETLSLTCAVTGYSITSGYSWHWIRQFPGKGLEWIGSIHYSGGTNYNPSLKSRITISR |
| | heavy chain | DTSKNQFSLKLSSVTAADTAVYFCARMTTAPRYPFDYWGQGTLVTVSS |
| | variable region | |
| 27 | 10D1_11B | DVQLQEWGAGLLKPSETLSLTCAVYGYSITSGYSWHWIRQPPGKGLEWIGSIHYSGGTNYNPSLKSRVTISR |
| | heavy chain | DTSKNQFSLKLSSVTAADTAVYYCARMTTAPRYPFDYWGQGTLVTVSS |
| | variable region | |
| 28 | 10D1_c85v1 | DVQLQEWGAGLLKPSETLSLTCAVTGYSITSGYSWHWIRQFPGNGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 29 | 10D1_c85v2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARMTTAPRYPFDYWGQGTLVTVSS |
| | variable region | |
| 30 | 10D1_c85o1 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARETTAPRYPFDYWGQGTLVTVSS |
| | variable region | |
| 31 | 10D1_c85o2 | DVQLQEWGAGLLKPSETLSLTCAVTGYSITSGYSWHWIRQFPGKGLEWIGSIRYSGGTNYNPSLKSRITISR |
| | heavy chain | DTSKNQFSLKLGSVTAADTAVYFCARGTTAPRYPFDYWGQGTLVTVSS |
| | variable region | |
| 32 | 10D1_c87 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 33 | 10D1_c89 heavy | QVQLQESGPGLVKPSQTLSLTCTVSGYSITSGYSWHWIRQHPGKGLEWIGSIRYSGGTDYNPSLKSLVTISA |
| | chain variable | DTSKNQFSLKLSSVTAADTAVYYCARMTTAPWYPFDYWGQGTTVTVSS |
| | region | |
| 34 | 10D1_c90 heavy | QVQLQESGPGLVKPSQTLFLTCTVSGYSITSGYSWHWIRQHPGKGLEWIGSIRYSGGTDYNPSLKSLVTISV |
| | chain variable | DTSKNQFSLKLSSVTAADTAVYYCARMTTAPWYPFDYWGQGTTVTVSS |
| | region | |
| 35 | 10D1_c91 heavy | QVQLQESGPGLVKPSQTLSLTCTVSGYYITSGYSWHWIRQHPGKGLEWIGSIRYSGGTDYNPSLKSLATISA |
| | chain variable | DTSKNQFSLKLSSVTAADTAVYYCARMTTAPWYPFDYWGQGTAVTVSS |
| | region | |
| 36 | 10D1_c92 heavy | DVQLQEWGAGLLKPSETLSLTCAVTGYSITSGYSWHWIRQFPGNGLEWIGSIHYSGGTNYNPTLKSRITISR |
| | chain variable | DTSKNQFSLKLSSVTAADTAVYFCARMTTAPRYPFDYWGQGTLVTVSS |
| | region | |
| 37 | 10D1_c93 heavy | DVQLQEWGAGLLKPSETLSLTCAVTGYSITSGYSWHWIRQFPGNGLEWIGSIHYSGGTNYNPSLKSRITISR |
| | chain variable | DTSKNQFSLRLSSVTAADTAVYFCARMTTAPRYPFDYWGQGTLVTVSS |
| | region | |
| 38 | 10D1, | GYSITSGYS |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c85o1 | |
| | 10D1_c85o2, | |
| | 10D1_c87, | |
| | 10D1_c89, | |
| | 10D1_c90, | |
| | 10D1_c92, | |
| | 10D1_c93 heavy | |
| | chain CDR1 | |
| 39 | 10D1_c91 heavy | GYYITSGYS |
| | chain CDR1 | |
| 40 | 10D1 derived | GYX₁ITSGYS |
| | consensus | wherein X₁ = S or Y |
| | heavy chain | |
| | CDR1 | |
| 41 | 10D1, | IHYSGGT |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c87, | |
| | 10D1_c92, | |
| | 10D1_c93 heavy | |
| | chain CDR2 | |
| 42 | 10D1_c85v1, | IRYSGGT |
| | 10D1_c85v2, | |
| | 10D1_c85o1, | |
| | 10D1_c85o2, | |
| | 10D1_c89, | |
| | 10D1_c90, | |
| | 10D1_c91 heavy | |
| | chain CDR2 | |
| 43 | 10D1 derived | IX₂YSGGT |
| | consensus | wherein X₂ = H or R |
| | heavy chain | |
| | CDR2 | |
| 44 | 10D1, | ARMTTAPRYPFDY |
| | 10D1_c75, | |
| | 10D1_c76, | |
| | 10D1_c77, | |
| | 10D1_c78v1, | |
| | 10D1_c78v2, | |
| | 10D1_11B, | |
| | 10D1_c85v1, | |
| | 10D1_c85v2, | |
| | 10D1_c87, | |
| | 10D1_c92, | |
| | 10D1_c93 heavy | |
| | chain CDR3 | |
| 45 | 10D1_c89, | ARMTTAPWYPFDY |
| | 10D1_c90, | |
| | 10D1_c91 heavy | |
| | chain CDR3 | |
| 46 | 10D1_c85o1 | ARETTAPRYPFDY |
| | heavy chain | |
| | CDR3 | |
| 47 | 10D1_c85o2 | ARGTTAPRYPFDY |
| | heavy chain | |
| | CDR3 | |
| 48 | 10D1 derived | ARX₃TTAPX₄YPFDY |
| | consensus | wherein X₃ = M, E or G; X₄ = R or W |
| | heavy chain | |
| | CDR3 | |
| 49 | 10D1 light chain | DIVMTQSQKFMSTSVGDRVSVTCKASQIVGSNVAWYQQKPGQSPKPLIYSASYRYSGVPDRFTASGSGTDFT |
| | variable region | LTITNVQSEDLAEYFCQQYSSHPLTFGAGTKLELK |
| 50 | 10D1_c75 light | DIVMTQSPSSLSASVGDLVTITCKASQIVGSNVAWYQMKPGKSPKPLIYSASYLYFGVPSRFSGSGSGTDFT |
| | chain variable | LTISSLQPEDVAEYFCQQYSSHPLTFGPGTKVEIK |
| | region | |
| 51 | 10D1_c76 light | DIVMTQSPSSLSASGGDRVTITCKASQIVGYNVAWYQQKPGKSPKPLIYSASYLYSDVPSRFSASGSGTDFT |
| | chain variable | LTISSLQPEDVAEYFCQQYSSHPLTFGPGTKVEIK |
| | region | |
| 52 | 10D1_c77 light | VIVMTQSPSSLSASVGDRVTITCKASQIVGPNVAWYQQKPGKSPKPLIYSASYGYSDVPSRFSGSGSGTDFT |
| | chain variable | LTISSLQPEDVAEYFCQQYSTHPLTFGPGTKVEIK |
| | region | |
| 53 | 10D1_c78v1, | DIVMTQSPSSLSASVGDRVTITCKASQIVGSNVAWYQQKPGKSPKPLIYSASYGYSDVPSRFSGSGSGTDFT |
| | 10D1_c78v2, | LTISSLRPEDVATYYCQQYSSHPLTFGPGTKVEIK |
| | 10D1_11B light | |
| | chain variable | |
| | region | |
| 54 | 10D1_c85v1, | DIVMTQSPSSLSASVGDRVTITCKASQIVGSNVAWYQQKPGKSPKPLIYSARYQYSGVPFRFSGSGSGTDFT |
| | 10D1_c85v2 | LTISSLQPEDVATYYCQQYSSHPLTFGPGTKVEIK |
| | light chain | |
| | variable region | |
| 55 | 10D1_c85o1 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 56 | 10D1_c85o2 | DIVMTQSPSSLSASVGDRVTITCKASQIVGSNVAWYQQKPGKSPKPLIYSARYQYSGVPFRFSGSGSGTDFT |
| | light chain | LTISSLQPEDVATYYCQQYSSHPLTFGPGTKVEIK |
| | variable region | |
| 57 | 10D1_c87 light | DIVMTQSPSSLSASVGDRVTITCKASQIVGSNVAWYQQMPGKSPEPLIYSASYLYSDVPSRFSGSGSGTDFT |
| | chain variable | MTISSLQPEDVATYYCQQYSSHPLTFGPGTKVEIK |
| | region | |
| 58 | 10D1_c89 light | DIQMTQSPSSVSASVGDRVTITCKASQIVGSNVAWYQQKPGKAPEPLIYSASYLYSGVPSRFSGSGSGTDFT |
| | chain variable | LTISSLQPEDFATYYCQQYSSHPLTFGQGTKLEIK |
| | region | |
| 59 | 10D1_c90 light | DIQMTQSPSSVSASVGDRVTFTCKASQIVGSNVAWYQQKPGKAPEPLIYSASYLYSSVPSRFSGSGSGTEFT |
| | chain variable | MTISSLEPEDFATYYCQQYTTHPLTFGPGTKVEIK |
| | region | |
| 60 | 10D1_c91 light | DIQMTQSPSSVSASVGDRVTITCKASQIVGSNVAWYQQKPGKAPMPLIYSASYGYSGVPSRFSGSGSGTDFT |
| | chain variable | LTISSLQPEDFATYYCQQYSSHPLTFGQGTKLEIK |
| | region | |
| 61 | 10D1_c92 light | DIVMTQSPSSLSASVGDLVTITCKASQIVGSNVAWYQMKLGKSPKPLIYSASYLYFGVPSRFSGSGSGTDFT |
| | chain variable | LTISSLQPEDVAEYFCQQYFSHPLTFGPGTKVEIK |
| | region | |
| 62 | 10D1_c93 light | DIVMTQSPSSLSASVGDRVTITCKASQIVGSNVAWYQQKPGKSPKPLIYSASYLYSDVPSRFSGSGSGTDFT |
| | chain variable | MTISSLQPEDVATYYCQQYSSHPLTFGPGTKVEIK |
| | region | |
| 63 | 10D1, | QIVGSN |
| | 10D1_c75, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89 | |
| | 10D1_c90, | |
| | 10D1_c91, | |
| | 10D1_c92, | |
| | 10D1_c93 light | |
| | chain CDR1 | |
| 64 | 10D1_c76 light | QIVGYN |
| | chain CDR1 | |
| 65 | 10D1_c77 light | QIVGPN |
| | chain CDR1 | |
| 66 | 10D1 derived | QIVGX₅N |
| | consensus light | wherein X₅ = S, Y or P |
| | chain CDR1 | |
| 67 | 10D1, | SAS |
| | 10D1_c75, | |
| | 10D1_c76, | |
| | 10D1_c77 | |
| | 10D1_c78v1, | |
| | 10D1_c78v2, | |
| | 10D1_11B, | |
| | 10D1_c87, | |
| | 10D1_c89 | |
| | 10D1_c90, | |
| | 10D1_c91, | |
| | 10D1_c92, | |
| | 10D1_c93 | |
| | light chain CDR2 | |
| 68 | 10D1_c85v1, | SAR |
| | 10D1_c85v2, | |
| | 10D1_c85o1, | |
| | 10D1_c85o2 | |
| | light chain CDR2 | |
| 69 | 10D1 derived | SAX₆ |
| | consensus light | wherein X₆ = S or R |
| | chain CDR2 | |
| 70 | 10D1, | QQYSSHPLT |
| | 10D1_c75, | |
| | 10D1_c76, | |
| | 10D1_c78v1, | |
| | 10D1_c78v2, | |
| | 10D1_11B, | |
| | 10D1_c85v1, | |
| | 10D1_c85v2, | |
| | 10D1_c85o1, | |
| | 10D1_c85o2, | |
| | 10D1_c87, | |
| | 10D1_c89, | |
| | 10D1_c91, | |
| | 10D1_c93 light | |
| | chain CDR3 | |
| 71 | 10D1_c77 light | QQYSTHPLT |
| | chain CDR3 | |
| 72 | 10D1_c90 light | QQYTTHPLT |
| | chain CDR3 | |
| 73 | 10D1_c92 light | QQYFSHPLT |
| | chain CDR3 | |
| 74 | 10D1 derived | QQYX₇X₈HPLT |
| | consensus light | wherein X₇ = S, T or F; X₈ = S or T |
| | chain CDR3 | |
| 75 | 10D1F hIgG1 | QVQLQESGPGLVKPSQTLSLTCTVSGYSITSGYSWHWIRQHPGKGLEWIGSIRYSGGTDYNPSLKSLVTISA |
| | HC | DTSKNQFSLKLSSVTAADTAVYYCARMTTAPWYPFDYWGQGTTVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAKGQPREPQVYTLPPSR |
| | | DELTKNQVSLTCLVKGFYPSDIAVEWESNGQPENNYKTTPPVLDSDGSFFLYSKLTVDKSRWQQGNVFSCS |
| | | VMHEALHNHYTQKSLSLSPGK |
| 76 | 10D1F K LC | DIQMTQSPSSVSASVGDRVTITCKASQIVGSNVAWYQQKPGKAPEPLIYSASYLYSGVPSRFSGSGSGTDFT |
| | | LTISSLQPEDFATYYCQQYSSHPLTFGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYACEVTHQGLSSPVTKSFNRGEC |

The present disclosure includes combinations of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may preferably be performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIGS. 1A and 1B. Bar chart and graph showing comparison of anti-cancer efficacy of 10D1F/10D1 P for different cancer cell lines determined by analysis in in vitro and in vivo studies. (1A) shows the efficacy of 10D1F/10D1P to inhibit growth of cells of the indicated cancer cell lines in vitro, or in vivo in cell-line derived xenograft models. (1B) shows a linear regression modelling the relationship between efficacy determined for treatment of the cell lines with 10D1F/10D1 P in vitro and in vivo.

FIGS. 2A and 2B. Graphs showing the response of cancer cell lines having different genotypes (see Example 2) to treatment with (2A) 10D1F/10D10P, and (2B) seribantumab, as determined by analysis in in vitro and in vivo studies. ****P≤0.0001.

FIGS. 3A and 3B. Graph showing the response of cancer cell lines having different genotypes (see Example 3) to treatment with (3A) 10D1F/10D1P, and (3B) seribantumab, as determined by analysis in in vitro and in vivo studies. ****P≤0.0001.

### EXAMPLES

In the present Examples the inventors demonstrate that extent of response to treatment with anti-HER3 antibodies is dependent on the mutational status of the cancer in genes encoding regulators of HER3-mediated signalling. They show that cancers lacking mutation to KRAS, PIK3CA, BRAF and PTEN respond exceptionally well to treatment with anti-HER3 antibodies. The Examples further show that NRG1 expression is predictive of a positive response to anti-HER3 antibody therapy.

### Example 1: Analysis of Correlation Between Inhibition of Cell Proliferation In Vitro and Tumor Growth Inhibition In Vivo

The inventors investigated whether the results of analysis of the ability of anti-HER3 antibody 10D1F hIgG1 (comprising VH=SEQ ID NO:33, VL=SEQ ID NO:58) or 10D1P hIgG1 (comprising VH=SEQ ID NO:21, VL=SEQ ID NO:49) to inhibit growth of cells of cancer cell lines in vitro is predictive of its ability to inhibit tumor growth of cell line-derived xenograft tumors in vivo.

The cell lines analysed were N87 (gastric cancer), FaDu (head and neck cancer), OvCAR8 (ovarian cancer), SNU16 (gastric cancer), A549 (lung cancer), HCC95 (lung cancer), AHCN (kidney cancer) and 22Rv1 (prostate cancer).

For analysis of inhibition of growth in vitro, cells of the different cell lines were treated with 10-point serially diluted concentrations of anti-HER3 antibody (3-fold dilations, starting from a maximum concentration of 1500 μg/ml), in triplicate. Cell viability was measured using CCK-8 assay (Dojindo) after 3-5 days. The percentage inhibition of growth of the cells was calculated by comparison to the CCK-8 assay signal for cells treated with buffer only (PBS). In vitro efficacy was determined to be the percentage inhibition observed at 1500 μg/ml.

For analysis of inhibition of growth in vivo, cell line-derived xenograft models were established by subcutaneous injection of cells into the right flank of female NCr nude mice (approximately 6-8 weeks old). Anti-HER3 antibody was administered by intraperitoneal injection, twice weekly at 25 mg/kg bodyweight per dose. Control treatment groups received an equal volume of PBS. Tumor volumes were measured 3 times a week using a digital caliper and calculated using the formula [L×W2/2]. Study End point was considered to have been reached once the tumors of the control arm measured ≥1.5 cm in length. In vivo efficacy was determined to be the tumor growth inhibition percentage at Study End.

Efficacy data from in vitro and in vivo studies were normalised to set the maximum inhibition as 100% and minimum inhibition as 0%, where the values were above or below this range. In vitro-in vivo efficacy correlation was studied by a simple linear regression and the correlation coefficient (R²) was calculated using GraphPad Prism software.

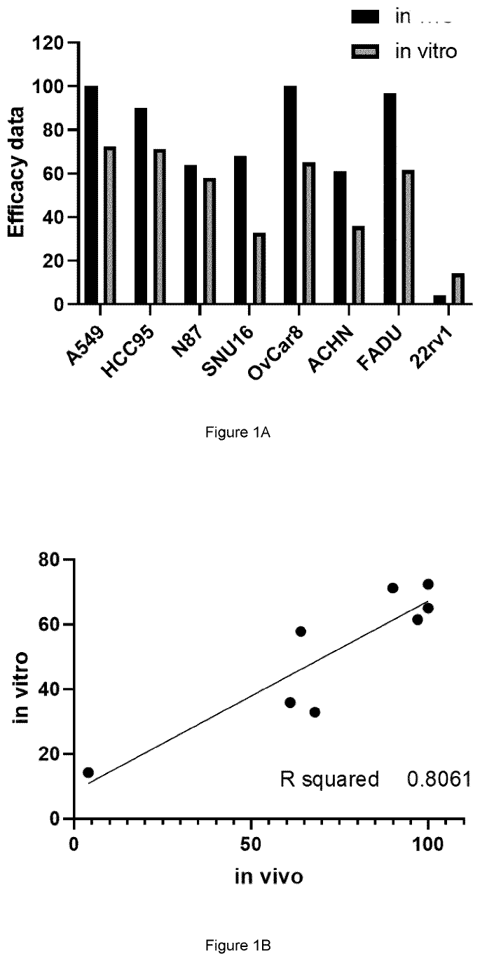

The results of the analysis are shown in FIGS. 1A and 1B. There was a strong correlation between results obtained in in vitro studies and in vivo studies (R²=0.8061). This indicates that the results of in vitro analyses of inhibition of cancer cell growth are predictive of efficacy of tumor growth inhibition in vivo (and vice versa).

### Example 2: Analysis of Efficacy of HER3-Binding Antigen-Binding Molecules to Treat Cancer Based on Mutation Status of Regulators of HER3-Mediated Signalling

The inventors employed a combination of data from in vivo and in vitro studies of the anti-cancer activity of 10D1F/10D1P or seribantumab to investigate whether the mutation status of the cancer cell lines was predictive of efficacy of treatment using the antibody.

Cell lines from breast, gastric and non-small cell lung cancers, colorectal cancer & SCCHN and prostate cancers were included in the analysis. The cell lines were analysed for presence of point mutations in KRAS, PIK3CA and for PTEN loss using public databases such as COSMIC, Cellosaurus and CCLE. Cell lines were categorised as: (i) wildtype for KRAS, PIK3CA and PTEN (‘wildtype’); (ii) heterozygous for an activating mutation to KRAS or PIK3CA, or heterozygous for an inactivating mutation to PTEN (‘heterozygous’); (iii) homozygous for an activating mutation to KRAS or PIK3CA, or homozygous for an inactivating mutation to PTEN (‘homozygous’).

In scoring responses to treatment for 10D1F/10D1P, tumor growth inhibition (TGI) data from in vivo studies using 10D1F hIgG1 were prioritised. Where such data were not available, TGI data from 10D1P hIgG1 were used. Where in vivo data was not available for a cell line in vitro inhibition data was used.

The relationship between normalised functional responses to treatment with 10D1F/10D1P or seribantumab and mutational status was analysed using GraphPad Prism. For comparisons between wildtype, heterozygous and homozygous groups, an unpaired t-test was performed.

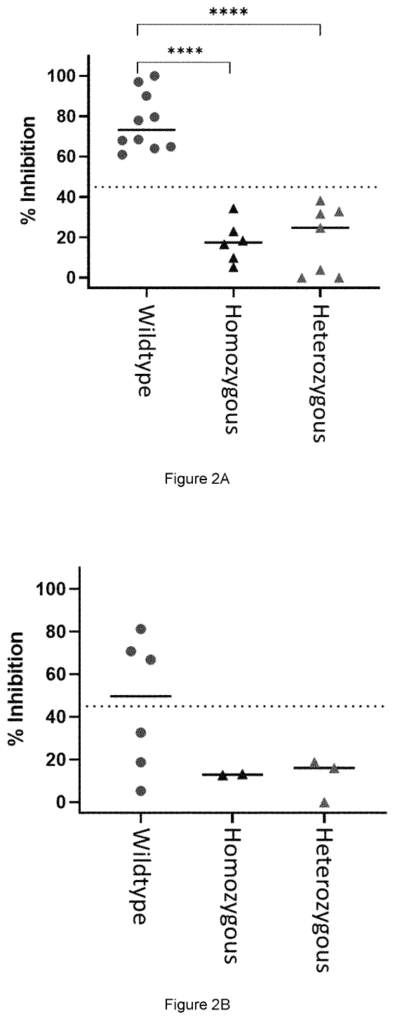

The genotypes of the cell lines for KRAS, PIK3CA and PTEN and their normalised functional responses to treatment with 10D1F/10D1P or seribantumab (as determined by analysis in in vitro or in vivo studies as described in Example 1) are shown in the table below, and the data are represented graphically in FIGS. 2A and 2B.

| | | | | | 10D1F/10D1P | seribantumab |
|---|---|---|---|---|---|---|
| Cell Line | KRAS | PIK3CA | PTEN | Category | response | response |
| FaDu | WT/WT | WT/WT | WT/WT | Wildtype | 97 (10D1F, in vivo) | 81.2 (in vivo) |
| HCC95 | WT/WT | WT/WT | WT/WT | Wildtype | 90 (10D1P, in vivo) | 70.8 (in vitro) |
| N87 | WT/WT | WT/WT | WT/WT | Wildtype | 64 (10D1F, in vivo) | 18.7 (in vivo) |
| SNU16 | WT/WT | WT/WT | WT/WT | Wildtype | 68 (10D1P, in vivo) | 32.57 (in vitro) |
| OvCar8 | WT/WT | WT/WT | WT/WT | Wildtype | 100 (10D1F, in vivo) | 66.8 (in vivo) |
| ACHN | WT/WT | WT/WT | WT/WT | Wildtype | 61 (10D1P, in vivo) | 5.3 (in vitro) |
| BCPAP | WT/WT | WT/WT | WT/WT | Wildtype | 64.87 (10D1F, in vitro) | NT |
| BHT101 | WT/WT | WT/WT | WT/WT | Wildtype | 68.5 (10D1F, in vitro) | NT |
| LoVo | G12C/WT | WT/WT | WT/WT | Heterozygous | 31.67 (10D1F, in vivo) | NT |
| HCT15 | G12D/WT | D549N, | | Heterozygous | 32.87 (10D1F, in vivo) | NT |
| | | E545K/WT | | | | |
| HCT116 | G12D/WT | H1047R/WT | | Heterozygous | 24.73 (10D1F, in vivo) | NT |
| HT29 | WT/WT | P449T/WT | | Heterozygous | 0 (10D1P, in vivo) | 16 (in vitro) |
| DU145 | WT/WT | | KO/WT | Heterozygous | 38.3 (10D1F, in vitro) | 18.7 (in vitro) |
| 22Rv1 | | Q546R/WT | | Heterozygous | 4 (10D1F, in vivo) | 0 (in vitro) |
| H358 | G12C/WT | | | Heterozygous | 0 (10D1F, in vitro) | NT |
| PC3 ATCC | | | KO/KO | Homozygous | 23 (10D1F, in vitro) | 13.12 (in vitro) |
| LnCap | | | KO/KO | Homozygous | 9.93 (10D1F, in vitro) | 12.7 (in vitro) |
| H1373 | G12C/G12C | | | Homozygous | 16.6 (10D1F, in vitro) | NT |
| H2030 | G12C/G12C | | | Homozygous | 18.4 (10D1F, in vitro) | NT |
| H2122 | G12C/G12C | | | Homozygous | 5.3 (10D1F, in vitro) | NT |
| FTC133 | | | KO/KO | Homozygous | 34.33 (10D1F, in vitro) | NT |

NT = not tested

The analysis revealed a very striking pattern, in which the response to treatment with anti-HER3 antibody was dependent on the presence of mutations resulting in upregulation of HER3-mediated signalling. Cell lines in the wildtype group were much more responsive to anti-HER3 antibody treatment compared to cell lines in the heterozygous or homozygous groups. This pattern was observed across the full range of different cancer types analysed.

### Example 3: Analysis of Efficacy of HER3-Binding Antigen-Binding Molecules to Treat Cancer Based on the Mutation Status of PTEN/KRAS/PIK3CA/BRAF and NRG1 mRNA Abundance

In further studies, the inventors employed the data from in vivo and in vitro studies of the anti-cancer activity of 10D1F/10D1P or seribantumab to investigate whether the mutation status of BRAF could be added to the triple gene signature (PTEN/KRAS/PIK3CA) described in Example 2 to predict the efficacy of anti-HER3 antibody treatment.

Cell lines from breast, gastric and non-small cell lung cancers, colorectal cancer & SCCHN and prostate cancers were included in the analysis. The cell lines were evaluated by empirical sequence analysis for the presence of point mutations in KRAS, PIK3CA and BRAF, and for PTEN loss. Cell lines were categorised as: (i) wildtype for KRAS, PIK3CA, PTEN, and BRAF (‘wildtype’); (ii) heterozygous for an activating mutation to KRAS or P1K3CA or BRAF, or heterozygous for an inactivating mutation to PTEN (‘heterozygous’); (iii) homozygous for an activating mutation to KRAS or PIK3CA or BRAF, or homozygous for an inactivating mutation to PTEN (‘homozygous’). Expression levels of NRG1 mRNA were also measured for each cell line.

In scoring responses to treatment for 10D1F/10D1P, tumor growth inhibition (TGI) data from in vivo studies using 10D1F hIgG1 were prioritised. Where such data were not available, TGI data from 10D1P hIgG1 were used. Where in vivo data was not available for a cell line in vitro inhibition data was used.

The relationship between normalised functional responses to treatment with 10D1F/10D1P or seribantumab and mutational status was analysed using GraphPad Prism. For comparisons between wildtype, heterozygous and homozygous groups, an unpaired t-test was performed.

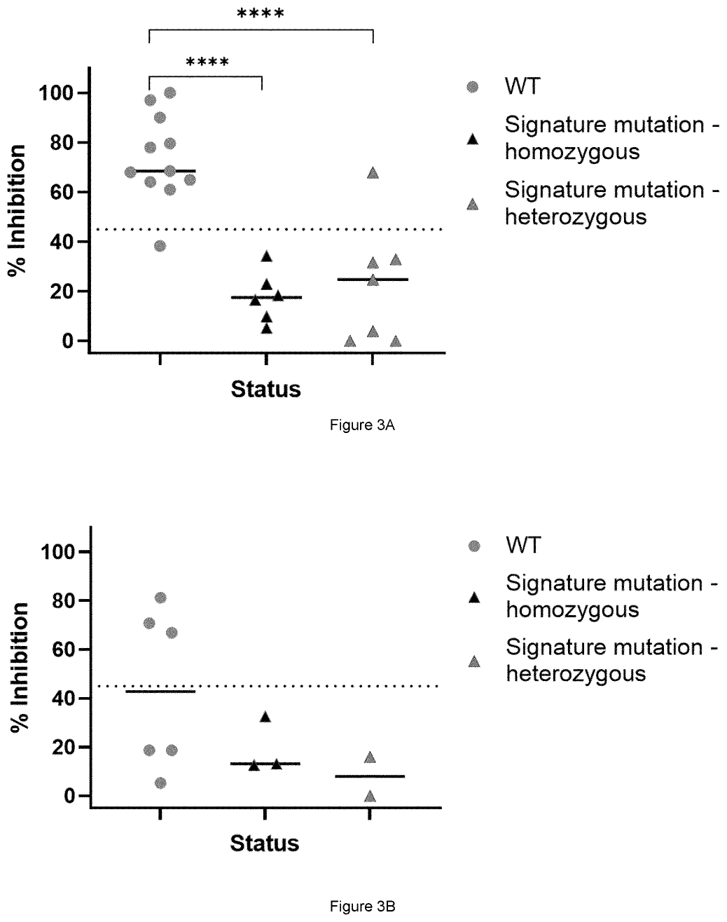

The genotypes of the cell lines for KRAS, PIK3CA, PTEN and BRAF as determined by sequence analysis and their normalised functional responses to treatment with 10D1F/10D1P orseribantumab (as determined by analysis in in vitro or in vivo studies as described in Example 1) are shown in the table below, and the data are represented graphically in FIGS. 3A and 3B.

| | | | | | | | | NRG1 |
|---|---|---|---|---|---|---|---|---|
| | | | | | | | | mRNA |
| | | | | | | 10D1F/10D1P | Seribantumab | expression |
| Cell line | KRAS | PIK3CA | PTEN | BRAF | Category* | response | response | (RPKM) |
| FaDu | WT/WT | WT/WT | WT/WT | WT/WT | Wildtype | 97 (10D1F, in vivo) | 81.2 (in vivo) | 0.75 |
| HCC95 | WT/WT | WT/WT | WT/WT | WT/WT | Wildtype | 90 (10D1P, in vivo) | 70.8 (in vitro) | 157.38 |
| N87 | WT/WT | WT/WT | WT/WT | WT/WT | Wildtype | 64 (10D1F, in vivo) | 18.7 (in vivo) | 0.07 |
| SNU16 | G12D/WT | WT/WT | WT/WT | WT/WT | Heterozygous | 68 (10D1F, in vivo) | 32.57 (in vitro) | 0.012 |
| OvCar8 | VVT/WT | WT/WT | WT/WT | WT/WT | Wildtype | 100 (10D1F, in vivo) | 66.8 (in vivo) | 5.62 |
| ACHN | WVT/WT | WT/WT | WT/WT | WT/WT | Wildtype | 61 (10D1P, in vivo) | 5.3 (in vitro) | 11.64 |
| BCPAP | WT/WT | WT/WT | WT/WT | V600E/ | Heterozygous | 64.87 (10D1F, in vitro) | NT | 0.95 |
| | | | | WT | | | | |
| BHT101 | WT/WT | WT/WT | WT/WT | V600E/ | Heterozygous | 68.5 (10D1F, in vitro) | NT | 1.66 |
| | | | | WT | | | | |
| LoVo | G13C/WT | WT/WT | WT/WT | WT/WT | Heterozygous | 31.67 (10D1F, in vivo) | NT | 0.0084 |
| HCT15 | G13D/WT | D549N, | WT/WT | WT/WT | Heterozygous | 32.87 (10D1F, in vivo) | NT | 0.014 |
| | | E545K/WT | | | | | | |
| HCT116 | G13D/WT | H1047R/WT | WT/WT | WT/WT | Heterozygous | 24.73 (10D1F, in vivo) | INT | 0.77 |
| HT29 | WT/WT | P449T/WT | WT/WT | V600E/ | Heterozygous | 0 (10D1P, in vivo) | 16 (in vitro) | 0.0038 |
| | | | | WT; | | | | |
| | | | | T119S/ | | | | |
| | | | | WT | | | | |
| DU145 | WT/WT | WT/WT | WT/WT | WT/WT | Wildtype | 38.3 (10D1F, in vitro) | 18.7 (in vitro) | 0.43 |
| 22Rv1 | WT/WT | Q546R/WT | WT/WT | L597R/ | Heterozygous | 4 (10D1F, in vivo) | 0 (in vitro) | 0.012 |
| | | | | WT | | | | |
| H358 | G12C/WT | WT/WT | KO/KO | WT/WT | Heterozygous | 0 (10D1F, in vitro) | NT | 3.32 |
| PC3 | WT/WT | WT/WT | KO/KO | WT/WT | Homozygous | 23 (10D1F, in vitro) | 13.12 (in vitro) | 0.022 |
| ATCC | | | | | | | | |
| LnCap | WT/WT | WT/WT | KO/KO | WT/WT | Homozygous | 9.93 (10D1F, in vitro) | 12.7 (in vitro) | 0.015 |
| H1373 | G12C/G12C | WT/WT | WT/WT | WT/WT | Homozygous | 16.6 (10D1F, in vitro) | NT | 1.44 |
| H2030 | G12C/G12C | WT/WT | WT/WT | WT/WT | Homozygous | 18.4 (10D1F, in vitro) | NT | 7.48 |
| H2122 | G12C/G12C | WT/WT | WT/WT | WT/WT | Homozygous | 5.3 (10D1F, in vitro) | NT | 0.64 |
| FTC133 | WT/WT | WT/WT | KO/KO | WT/WT | Homozygous | 34.33 (10D1F, in vitro) | NT | 2.54 |

NT = Not tested

The analysis revealed that the mutational status of BRAF in combination with PIK3CA, KRAS, and PTEN is predictive for the efficacy of anti-HER3 antibody treatment, and cell lines in the wildtype group were much more responsive to anti-HER3 antibody treatment compared with cell lines from heterozygous or homozygous groups (see FIGS. 3A and 3B). This pattern was observed across a wide range of preclinical cancer models.

The data in the table above further demonstrate a link between NRG1 expression and treatment efficacy, with increased NRG1 expression correlating with improved efficacy of anti-HER3 antibody treatment.

In conclusion, a gene signature comprising the mutational status of PIK3CA, KRAS, BRAF, and PTEN consistently and significantly predicts the efficacy of anti-HER3 antibody treatment in preclinical cancer models, and this gene signature can be combined with NRG1 mRNA abundance as an additional predictive factor.

1. An antigen-binding molecule which binds to HER3 for use in a method of treating or preventing a HER3-associated cancer in a subject, wherein the HER3-associated cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

2. Use of an antigen-binding molecule which binds to HER3 in the manufacture of a medicament for use in treating or preventing a HER3-associated cancer in a subject, wherein the HER3-associated cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

3. A method of treating or preventing a HER3-associated cancer in a subject, comprising administering to the subject a therapeutically- or prophylactically-effective amount of an antigen-binding molecule which binds to HER3, wherein the HER3-associated cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation.

4. The antigen-binding molecule for use according to claim 1, the use according to claim 2, or the method according to claim 3, wherein the HER3-associated cancer: (i) does not comprise an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF; or (ii) does not comprise an inactivating mutation to a gene selected from: PTEN, PPP2CA, PIK3R1, PIK3R2, NF1, BAD and PHLPP1.

5. The antigen-binding molecule for use, the use, or the method according to any one of claims 1 to 4, wherein the HER3-associated cancer: (i) does not comprise an activating mutation to KRAS; or (ii) does not comprise an activating mutation to PIK3CA; or (iii) does not comprise an activating mutation to BRAF; or (iv) does not comprise an inactivating mutation to PTEN.

6. The antigen-binding molecule for use, the use, or the method according to any one of claims 1 to 5, wherein the HER3-associated cancer: (i) does not comprise an activating mutation to KRAS and does not comprise an activating mutation to PIK3CA; or (ii) does not comprise an activating mutation to KRAS and does not comprise an inactivating mutation to PTEN; or (iii) does not comprise an activating mutation to PIK3CA and does not comprise an inactivating mutation to PTEN; or (iv) does not comprise an activating mutation to KRAS and does not comprise an activating mutation to BRAF; or (v) does not comprise an activating mutation to PIK3CA and does not comprise an activating mutation to BRAF; or (vi) does not comprise an activating mutation to BRAF and does not comprise an inactivating mutation to PTEN; or (vii) does not comprise an activating mutation to KRAS and does not comprise an activating mutation to PIK3CA and does not comprise an activating mutation to BRAF; or (viii) does not comprise an activating mutation to PIK3CA and does not comprise an activating mutation to BRAF and does not comprise an inactivating mutation to PTEN; or (ix) does not comprise an activating mutation to BRAF and does not comprise an activating mutation to KRAS and does not comprise an inactivating mutation to PTEN; or (x) does not comprise an activating mutation to KRAS and does not comprise an activating mutation to PIK3CA, and does not comprise an inactivating mutation to PTEN; or (xi) does not comprise an activating mutation to KRAS and does not comprise an activating mutation to BRAF and does not comprise an activating mutation to PIK3CA and does not comprise an inactivating mutation to PTEN.

7. The antigen-binding molecule for use, the use, or the method according to any one of claims 1 to 6, wherein the HER3-associated cancer does not comprise a mutation resulting in upregulation of HER3-mediated signalling.

8. The antigen-binding molecule for use, the use, or the method according to any one of claims 1 to 7, wherein the HER3-associated cancer comprises cells expressing NRG1 at a level which is greater than the level of expression by equivalent non-cancerous cells.

9. An antigen-binding molecule which binds to HER3 for use in a method of treating or preventing a HER3-associated cancer in a subject, wherein the HER3-associated cancer comprises a mutation resulting in upregulation of HER3-mediated signalling, and wherein the method further comprises administering an antagonist of HER3-mediated signalling.

10. Use of an antigen-binding molecule which binds to HER3 in the manufacture of a medicament for use in treating or preventing a HER3-associated cancer in a subject, wherein the HER3-associated cancer comprises a mutation resulting in upregulation of HER3-mediated signalling, and wherein the method further comprises administering an antagonist of HER3-mediated signalling.

11. A method of treating or preventing a HER3-associated cancer in a subject, comprising administering to the subject a therapeutically- or prophylactically-effective amount of an antigen-binding molecule which binds to HER3, wherein the HER3-associated cancer comprises a mutation resulting in upregulation of HER3-mediated signalling, and wherein the method further comprises administering an antagonist of HER3-mediated signalling.

12. The antigen-binding molecule for use according to claim 9, the use according to claim 10, or the method according to claim 11, wherein the mutation resulting in upregulation of HER3-mediated signalling is an activating mutation to a gene encoding a positive regulator of HER3-mediated signalling or an inactivating mutation to a gene encoding a negative regulator of HER3-mediated signalling.

13. The antigen-binding molecule for use, the use, or the method according to any one of claims 9 to 12, wherein the HER3-associated cancer comprises: (i) an activating mutation to a gene selected from: KRAS, PIK3CA, PIK3CB, PIK3CD, ERBB3, ERBB2, ERBB4, EGFR, IGF1R, NRG1, NRG2, EGF, IRS2, GRB2, GAB2, PTPN11, SHP2, SOS1, HRAS, NRAS, RAF1, MAP2K1, MAP2K2, MAPK1, MYC, RPS6KA1, RPS6, MKNK1, CREB1, MTOR, PDK1, AKT1, AKT2, AKT3, JAK2, STAT3, STAT5, and BRAF; and/or (ii) an inactivating mutation to a gene selected from: PTEN, PPP2CA, PIK3R1, PIK3R2, NF1, BAD and PHLPP1.

14. The antigen-binding molecule for use, the use, or the method according to any one of claims 9 to 13, wherein the HER3-associated cancer comprises one or more of: an activating mutation to KRAS, an activating mutation to PIK3CA, an activating mutation to BRAF, or an inactivating mutation to PTEN.

15. A method of selecting a subject for treatment with an antigen-binding molecule which binds to HER3, comprising:
(a) analysing a subject's cancer in order to determine whether the cancer: (i) comprises at least one gene encoding a positive regulator of HER3-mediated signalling that does not comprise an activating mutation; or (ii) comprises at least one gene encoding a negative regulator of HER3-mediated signalling that does not comprise an inactivating mutation; and
(b) selecting a subject for treatment with an antigen-binding molecule which binds to HER3 where the subject's cancer is determined in step (a) not to comprise such a mutation.

16. The method according to claim 15, wherein the method further comprises:
(c) administering an antigen-binding molecule which binds to HER3 to a subject selected for treatment in step (b).

17. A method of selecting a subject for treatment with (i) an antagonist of HER3-mediated signalling and (ii) an antigen-binding molecule which binds to HER3, comprising:
(a) analysing a subject's cancer in order to determine whether the cancer comprises a mutation resulting in upregulation of HER3-mediated signalling; and
(b) selecting a subject for treatment with (i) an antagonist of HER3-mediated signalling and (ii) an antigen-binding molecule which binds to HER3 where the subject's cancer is determined in step (a) to comprise such a mutation.

18. The method according to claim 17, wherein the method further comprises:
(c) administering (i) an antagonist of HER3-mediated signalling and (ii) an antigen-binding molecule which binds to HER3, to a subject selected for treatment in step (b).

19. The antigen-binding molecule for use, the use, or the method according to any one of claims 1 to 18, wherein the HER3-associated cancer is selected from: a solid tumor, breast cancer, breast carcinoma, ductal carcinoma, gastric cancer, gastric carcinoma, gastric adenocarcinoma, colorectal cancer, colorectal carcinoma, colorectal adenocarcinoma, head and neck cancer, squamous cell carcinoma of the head and neck, lung cancer, non-small cell lung cancer, lung adenocarcinoma, squamous cell lung carcinoma, ovarian cancer, ovarian carcinoma, ovarian serous adenocarcinoma, renal cancer, renal cell carcinoma, renal clear cell carcinoma, renal cell adenocarcinoma, renal papillary cell carcinoma, pancreatic cancer, pancreatic adenocarcinoma, pancreatic ductal adenocarcinoma, cervical cancer, cervical squamous cell carcinoma, skin cancer, melanoma, esophageal cancer, esophageal adenocarcinoma, liver cancer, hepatocellular carcinoma, cholangiocarcinoma, uterine cancer, uterine corpus endometrial carcinoma, thyroid cancer, thyroid carcinoma, pheochromocytoma, paraganglioma, bladder cancer, bladder urothelial carcinoma, prostate cancer, prostate adenocarcinoma, sarcoma and thymoma.

20. The antigen-binding molecule for use, the use, or the method according to any one of claims 1 to 19, wherein the antigen-binding molecule which binds to HER3 is selected from: 10D1F, seribantumab, elgemtumab, patritumab, GSK2849330, lumretuzumab, CDX-3379, AV-203, barecetamab, TK-A3, TK-A4, MP-EV20, 1A5-3D4, 9F7-F11, 16D3-C1, NG33, A5, F4, huHER3-8, REGN1400 and zenocutuzumab.

21. The antigen-binding molecule for use, the use, or the method according to any one of claims 1 to 20, wherein the antigen-binding molecule which binds to HER3 comprises:
(i) a heavy chain variable (VH) region incorporating the following CDRs:
HC-CDR1 having the amino acid sequence of SEQ ID NO:40
HC-CDR2 having the amino acid sequence of SEQ ID NO:43
HC-CDR3 having the amino acid sequence of SEQ ID NO:48; and
(ii) a light chain variable (VL) region incorporating the following CDRs:
LC-CDR1 having the amino acid sequence of SEQ ID NO:66
LC-CDR2 having the amino acid sequence of SEQ ID NO:69
LC-CDR3 having the amino acid sequence of SEQ ID NO:74.

22. The antigen-binding molecule for use, the use, or the method according to any one of claims 1 to 21, wherein the antigen-binding molecule comprises:
(i) a VH region incorporating the following CDRs:
HC-CDR1 having the amino acid sequence of SEQ ID NO:38
HC-CDR2 having the amino acid sequence of SEQ ID NO:42
HC-CDR3 having the amino acid sequence of SEQ ID NO:45; and
(ii) a VL region incorporating the following CDRs:
LC-CDR1 having the amino acid sequence of SEQ ID NO:63
LC-CDR2 having the amino acid sequence of SEQ ID NO:67
LC-CDR3 having the amino acid sequence of SEQ ID NO:70.

23. The antigen-binding molecule for use, the use, or the method according to any one of claims 1 to 22, wherein the antigen-binding molecule comprises:
a VH region comprising an amino acid sequence having at least 70% sequence identity to the amino acid sequence of SEQ ID NO:33; and
a VL region comprising an amino acid sequence having at least 70% sequence identity to the amino acid sequence of SEQ ID NO:58.

24. The antigen-binding molecule for use, the use, or the method according to any one of claims 1 to 2, wherein the antigen-binding molecule comprises:
a polypeptide comprising, or consisting of, an amino acid sequence having at least 70% sequence identity to the amino acid sequence of SEQ ID NO:75; and
a polypeptide comprising, or consisting of, an amino acid sequence having at least 70% sequence identity to the amino acid sequence of SEQ ID NO:76.


SEQUENCE LISTING (76 sequences)

SEQ ID NO: 1 - protein, 1342 aa, synthetic construct
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 2 - protein, 183 aa, synthetic construct
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 3 - protein, 331 aa, synthetic construct
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 4 - protein, 1283 aa, synthetic construct
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 5 - protein, 699 aa, synthetic construct
MALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 6 - protein, 1323 aa, synthetic construct
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 7 - protein, 164 aa, synthetic construct
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTGQFPMVPSGLTPQPAQDWYLLDDDPRLLTLSASSKVPVTLAAV

SEQ ID NO: 8 - protein, 312 aa, synthetic construct
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKAF

SEQ ID NO: 9 - protein, 624 aa, synthetic construct
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 10 - protein, 21 aa, synthetic construct
MALTVIAGLVVIFMMLGGTFL

SEQ ID NO: 11 - protein, 678 aa, synthetic construct
YWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 12 - protein, 44 aa, synthetic construct
YWRGRRIQNKRAMRRYLERGESIEPLDPSEKANKVLARIFKETE

SEQ ID NO: 13 - protein, 258 aa, synthetic construct
LRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFT

SEQ ID NO: 14 - protein, 376 aa, synthetic construct
RMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 15 - protein, 164 aa, synthetic construct
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSC

SEQ ID NO: 16 - protein, 146 aa, synthetic construct
PPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPK

SEQ ID NO: 17 - protein, 166 aa, synthetic construct
ACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVA

SEQ ID NO: 18 - protein, 148 aa, synthetic construct
EGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLT

SEQ ID NO: 19 - protein, 17 aa, synthetic construct
QPLVYNKLTFQLEPNPH

SEQ ID NO: 20 - protein, 1283 aa, synthetic construct
MGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWKDIVRDQDAEIVVKDNGRSCPLCHEVCKGRCWGPGPEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMYNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKIIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGEAFQESAVSGSSEWCPRPVSLHPMPRGCLASESSEGHVTGSEAELQEKVSTCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPRPPRPSSLEELGYEYMDVGSDLSASLGSTQSCPLHPVPVMPTAGTTPDEDYEYMNRQRGGSGPGGDYAAMGACPASEQGYEEMRAFQGPGHQAPHVHYAHLKTLRSLEATDSAFDNPDYWHSRLFPKANAQRT

SEQ ID NO: 21 - protein, 120 aa, synthetic construct
DVQLQESGPDLVKPSQSLSLTCTVTGYSITSGYSWHWIRQFPGNKLEWMGSIHYSGGTNYNPSLKSRISITRDTSKNQFFLQLNSVTTEDTATYFCARMTTAPRYPFDYWGQGTTLTVSS

SEQ ID NO: 22 - protein, 120 aa, synthetic construct
DVQLQEWGAGLLKPSETLSLTCAVTGYSITSGYSWHWIRQFPGNGLEWIGSIHYSGGTNYNPTLKSRITISRDTSKNQFSLKLSSVTAADTAVYFCARMTTAPRYPFDYWGQGTLVTVSS

SEQ ID NO: 23 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLKLSSVTAADTAVYFCARMTTAPRYPFDYWGQGTLVTVSS

SEQ ID NO: 24-25 - skipped (2 entries)

SEQ ID NO: 26 - protein, 120 aa, synthetic construct
DVQLQEWGAGLLKPSETLSLTCAVTGYSITSGYSWHWIRQFPGKGLEWIGSIHYSGGTNYNPSLKSRITISRDTSKNQFSLKLSSVTAADTAVYFCARMTTAPRYPFDYWGQGTLVTVSS

SEQ ID NO: 27 - protein, 120 aa, synthetic construct
DVQLQEWGAGLLKPSETLSLTCAVYGYSITSGYSWHWIRQPPGKGLEWIGSIHYSGGTNYNPSLKSRVTISRDTSKNQFSLKLSSVTAADTAVYYCARMTTAPRYPFDYWGQGTLVTVSS

SEQ ID NO: 28 - protein, 120 aa, synthetic construct
DVQLQEWGAGLLKPSETLSLTCAVTGYSITSGYSWHWIRQFPGNGLEWIGSIRYSGGTNYNPSLKSRITISRDTSKNQFSLKLGSVTAADTAVYFCARMTTAPRYPFDYWGQGTLVTVSS

SEQ ID NO: 29 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARMTTAPRYPFDYWGQGTLVTVSS

SEQ ID NO: 30 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARETTAPRYPFDYWGQGTLVTVSS

SEQ ID NO: 31 - protein, 120 aa, synthetic construct
DVQLQEWGAGLLKPSETLSLTCAVTGYSITSGYSWHWIRQFPGKGLEWIGSIRYSGGTNYNPSLKSRITISRDTSKNQFSLKLGSVTAADTAVYFCARGTTAPRYPFDYWGQGTLVTVSS

SEQ ID NO: 32 - protein, 120 aa, synthetic construct
DVQLQEWGAGLLKPSETLSLTCAVTGYSITSGYSWHWIRQFPGNGLEWIGSIHYSGGTNYNPSLKSRITISRDTSKNQFSLRLSSVTAADTAVYFCARMTTAPRYPFDYWGQGTLVTVSS

SEQ ID NO: 33 - protein, 120 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSS

SEQ ID NO: 34 - protein, 120 aa, synthetic construct
QVQLQESGPGLVKPSQTLFLTCTVSGYSITSGYSWHWIRQHPGKGLEWIGSIRYSGGTDYNPSLKSLVTISVDTSKNQFSLKLSSVTAADTAVYYCARMTTAPWYPFDYWGQGTTVTVSS

SEQ ID NO: 35 - protein, 120 aa, synthetic construct
QVQLQESGPGLVKPSQTLSLTCTVSGYYITSGYSWHWIRQHPGKGLEWIGSIRYSGGTDYNPSLKSLATISADTSKNQFSLKLSSVTAADTAVYYCARMTTAPWYPFDYWGQGTAVTVSS

SEQ ID NO: 36-37 - skipped (2 entries)

SEQ ID NO: 38 - protein, 9 aa, synthetic construct
GYSITSGYS

SEQ ID NO: 39 - protein, 9 aa, synthetic construct
GYYITSGYS

SEQ ID NO: 40 - protein, 9 aa, synthetic construct
  MOD_RES 3: note = S or Y
GYXITSGYS

SEQ ID NO: 41 - protein, 7 aa, synthetic construct
IHYSGGT

SEQ ID NO: 42 - protein, 7 aa, synthetic construct
IRYSGGT

SEQ ID NO: 43 - protein, 7 aa, synthetic construct
  MOD_RES 2: note = H or R
IXYSGGT

SEQ ID NO: 44 - protein, 13 aa, synthetic construct
ARMTTAPRYPFDY

SEQ ID NO: 45 - protein, 13 aa, synthetic construct
ARMTTAPWYPFDY

SEQ ID NO: 46 - protein, 13 aa, synthetic construct
ARETTAPRYPFDY

SEQ ID NO: 47 - protein, 13 aa, synthetic construct
ARGTTAPRYPFDY

SEQ ID NO: 48 - protein, 13 aa, synthetic construct
  MOD_RES 3: note = M, E or G
  MOD_RES 8: note = R or W
ARXTTAPXYPFDY

SEQ ID NO: 49 - protein, 107 aa, synthetic construct
DIVMTQSQKFMSTSVGDRVSVTCKASQIVGSNVAWYQQKPGQSPKPLIYSASYRYSGVPDRFTASGSGTDFTLTITNVQSEDLAEYFCQQYSSHPLTFGAGTKLELK

SEQ ID NO: 50 - protein, 107 aa, synthetic construct
DIVMTQSPSSLSASVGDLVTITCKASQIVGSNVAWYQMKPGKSPKPLIYSASYLYFGVPSRFSGSGSGTDFTLTISSLQPEDVAEYFCQQYSSHPLTFGPGTKVEIK

SEQ ID NO: 51 - protein, 107 aa, synthetic construct
DIVMTQSPSSLSASGGDRVTITCKASQIVGYNVAWYQQKPGKSPKPLIYSASYLYSDVPSRFSASGSGTDFTLTISSLQPEDVAEYFCQQYSSHPLTFGPGTKVEIK

SEQ ID NO: 52 - protein, 107 aa, synthetic construct
VIVMTQSPSSLSASVGDRVTITCKASQIVGPNVAWYQQKPGKSPKPLIYSASYGYSDVPSRFSGSGSGTDFTLTISSLQPEDVAEYFCQQYSTHPLTFGPGTKVEIK

SEQ ID NO: 53 - protein, 107 aa, synthetic construct
DIVMTQSPSSLSASVGDRVTITCKASQIVGSNVAWYQQKPGKSPKPLIYSASYGYSDVPSRFSGSGSGTDFTLTISSLRPEDVATYYCQQYSSHPLTFGPGTKVEIK

SEQ ID NO: 54 - protein, 107 aa, synthetic construct
DIVMTQSPSSLSASVGDRVTITCKASQIVGSNVAWYQQKPGKSPKPLIYSARYQYSGVPFRFSGSGSGTDFTLTISSLQPEDVATYYCQQYSSHPLTFGPGTKVEIK

SEQ ID NO: 55-56 - skipped (2 entries)

SEQ ID NO: 57 - protein, 107 aa, synthetic construct
DIVMTQSPSSLSASVGDRVTITCKASQIVGSNVAWYQQMPGKSPEPLIYSASYLYSDVPSRFSGSGSGTDFTMTISSLQPEDVATYYCQQYSSHPLTFGPGTKVEIK

SEQ ID NO: 58 - protein, 107 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIK

SEQ ID NO: 59 - protein, 107 aa, synthetic construct
DIQMTQSPSSVSASVGDRVTFTCKASQIVGSNVAWYQQKPGKAPEPLIYSASYLYSSVPSRFSGSGSGTEFTMTISSLEPEDFATYYCQQYTTHPLTFGPGTKVEIK

SEQ ID NO: 60 - protein, 107 aa, synthetic construct; identical to SEQ ID NO: 59

SEQ ID NO: 61 - protein, 107 aa, synthetic construct
DIVMTQSPSSLSASVGDLVTITCKASQIVGSNVAWYQMKLGKSPKPLIYSASYLYFGVPSRFSGSGSGTDFTLTISSLQPEDVAEYFCQQYFSHPLTFGPGTKVEIK

SEQ ID NO: 62 - protein, 107 aa, synthetic construct
DIVMTQSPSSLSASVGDRVTITCKASQIVGSNVAWYQQKPGKSPKPLIYSASYLYSDVPSRFSGSGSGTDFTMTISSLQPEDVATYYCQQYSSHPLTFGPGTKVEIK

SEQ ID NO: 63 - protein, 6 aa, synthetic construct
QIVGSN

SEQ ID NO: 64 - protein, 6 aa, synthetic construct
QIVGYN

SEQ ID NO: 65 - protein, 6 aa, synthetic construct
QIVGPN

SEQ ID NO: 66 - protein, 6 aa, synthetic construct
  MOD_RES 5: note = S, Y or P
QIVGXN

SEQ ID NO: 67-69 - skipped (3 entries)

SEQ ID NO: 70 - protein, 9 aa, synthetic construct
QQYSSHPLT

SEQ ID NO: 71 - protein, 9 aa, synthetic construct
QQYSTHPLT

SEQ ID NO: 72 - protein, 9 aa, synthetic construct
QQYTTHPLT

SEQ ID NO: 73 - protein, 9 aa, synthetic construct
QQYFSHPLT

SEQ ID NO: 74 - protein, 9 aa, synthetic construct
  MOD_RES 4: note = S, T or F
  MOD_RES 5: note = S or T
QQYXXHPLT

SEQ ID NO: 75 - protein, 450 aa, synthetic construct
QVQLQESGPGLVKPSQTLSLTCTVSGYSITSGYSWHWIRQHPGKGLEWIGSIRYSGGTDYNPSLKSLVTISADTSKNQFSLKLSSVTAADTAVYYCARMTTAPWYPFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
DIQMTQSPSSVSASVGDRVTITCKASQIVGSNVAWYQQKPGKAPEPLIYSASYLYSGVPSRFSGSGSGTDFTLTISSLQPEDFATYYCQQYSSHPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC